(19) United States
(12) Patent Application Publication
(10) Pub. No.: US 2024/0382274 A1
(43) Pub. Date: Nov. 21, 2024

(54) CONTINUUM ROBOT AND INFORMATION PROCESSING APPARATUS

(71) Applicant: CANON KABUSHIKI KAISHA, Tokyo (JP)

(72) Inventors: KEIGO AKIYA, Tokyo (JP); KEI SATO, Kanagawa (JP)

(21) Appl. No.: 18/785,593

(22) Filed: Jul. 26, 2024

Related U.S. Application Data
(63) Continuation of application No. PCT/JP2022/048446, filed on Dec. 28, 2022.

(30) Foreign Application Priority Data
Jan. 28, 2022 (JP) 2022-011643

(51) Int. Cl. A61B 34/30; A61M 25/01; G16H 20/40; G16H 40/63

(52) U.S. Cl. CPC A61B 34/30; G16H 20/40; G16H 40/63; A61B 2034/301; A61M 25/0113

(57) ABSTRACT

A medical device includes: a base unit including a driving source and a coupling portion connected to the driving source; a bendable unit including a bend portion configured to bend and a held portion coupled to the coupling portion and configured to move so as to bend the bend portion, the bendable unit being detachably attachable to the base unit; an operating portion movable between a fixed position where the coupling portion and the held portion are fixed to each other and a position where the coupling portion and the held portion are not fixed to each other, in a state where the bendable unit and the base unit are attached to each other; and fixation detection means configured to detect whether the operating portion is at the fixed position.

### CROSS-REFERENCE TO RELATED APPLICATIONS

This application is a Continuation of International Patent Application No. PCT/JP2022/048446, filed Dec. 28, 2022, which claims the benefit of Japanese Patent Application No. 2022-011643, filed Jan. 28, 2022, both of which are hereby incorporated by reference herein in their entireties.

### TECHNICAL FIELD

The present invention relates to a continuum robot, an information processing apparatus, a method, and a program.

### BACKGROUND ART

A medical device made up of a continuum robot is used. PTL 1 describes a medical tool. The medical tool includes an operated portion having a deformable portion and an operating portion that deforms the deformable portion. In the medical tool, the operated portion and the operating portion are detachably attachable to each other.

### CITATION LIST

### Patent Literature

PTL 1 Japanese Patent Laid-Open No. 2013-248117

In PTL 1, a user performs attaching/detaching work for the operating portion and the operated portion; however, there is no configuration to detect the situation of attaching/detaching work. For this reason, although attaching/detaching work is not properly completed, the user may proceed to the subsequent work and, as a result, a setup may be, for example, performed again.

### SUMMARY OF INVENTION

One or more aspects of the present disclosure have been contemplated in view of the above-described points, and it is at least one object of the present disclosure to assist in attaching/detaching work in a continuum robot configured such that a bendable unit is detachably attachable to a base unit.

A continuum robot according to the present invention includes: a base unit including a driving source and a coupling portion connected to the driving source; a bendable unit detachably attached to the base unit, the bendable unit including a bend portion configured to bend and a held portion coupled to the coupling portion and configured to move so as to bend the bend portion, the bendable unit being detachably attachable to the base unit; an operating portion movable between a fixed position where the coupling portion and the held portion are fixed to each other and a position where the coupling portion and the held portion are not fixed to each other, in a state where the bendable unit and the base unit are attached to each other; and fixation detection means configured to detect whether the operating portion is at the fixed position.

Further features of the present invention will become apparent from the following description of exemplary embodiments with reference to the attached drawings.

### BRIEF DESCRIPTION OF DRAWINGS

FIG. 1 is an overall view of a medical system.

FIG. 2 is a perspective view that shows a medical device and a support.

FIG. 3A is a view that illustrates a catheter.

FIG. 3B is a view that illustrates the catheter.

FIG. 4A is a view that illustrates a catheter unit.

FIG. 4B is a view that illustrates the catheter unit.

FIG. 5A is a view that illustrates a base unit and a wire drive portion.

FIG. 5B is a view that illustrates the base unit and the wire drive portion.

FIG. 5C is a view that illustrates the base unit and the wire drive portion.

FIG. 6A is a view that illustrates the wire drive portion, a coupling device, and a bend drive portion.

FIG. 6B is a view that illustrates the wire drive portion, the coupling device, and the bend drive portion.

FIG. 6C is a view that illustrates the wire drive portion, the coupling device, and the bend drive portion.

FIG. 7A is a view that illustrates attachment of the catheter unit.

FIG. 7B is a view that illustrates attachment of the catheter unit.

FIG. 8A is a view that illustrates coupling of the catheter unit with the base unit.

FIG. 8B is a view that illustrates coupling of the catheter unit with the base unit.

FIG. 9 is an exploded view that illustrates coupling of the catheter unit with the base unit.

FIG. 10 is a view that illustrates fixing of a drive wire by a coupling portion.

FIG. 11 is a view that illustrates fixing of the drive wire by the coupling portion.

FIG. 12 is a view that illustrates fixing of the drive wire by the coupling portion.

FIG. 13 is a view that illustrates fixing of the drive wire by the coupling portion.

FIG. 14 is a view that illustrates fixing of the drive wire by the coupling portion.

FIG. 15A is a view that illustrates the catheter unit.

FIG. 15B is a view that illustrates a button.

FIG. 15C is a view that illustrates the base unit.

FIG. 16A is a view that illustrates operations of an operating portion.

FIG. 16B is a view that illustrates operations of the operating portion.

FIG. 16C is a view that illustrates operations of the operating portion.

FIG. 17A is a view that illustrates operations of the operating portion.

FIG. 17B is a view that illustrates operations of the operating portion.

FIG. 17C is a view that illustrates operations of the operating portion.

FIG. 18A is a sectional view that illustrates operations of the operating portion.

FIG. 18B is a sectional view that illustrates operations of the operating portion.

FIG. 18C is a sectional view that illustrates operations of the operating portion.

FIG. 19 is a flowchart that shows a use procedure of the medical system.

FIG. 20 is a flowchart that shows checking of attachment of the catheter unit with the base unit according to a first embodiment.

FIG. 21 is a flowchart that shows checking of removal of the catheter unit from the base unit according to the first embodiment.

FIG. 22 is a flowchart that shows monitoring of attachment of the catheter unit with the base unit according to the first embodiment.

FIG. 23A is a view that illustrates attachment of a catheter unit according to a second embodiment.

FIG. 23B is a view that illustrates attachment of the catheter unit according to the second embodiment.

FIG. 24 is a flowchart that shows checking of attachment of the catheter unit with the base unit according to the second embodiment.

FIG. 25 is a flowchart that shows checking of removal of the catheter unit from the base unit according to the second embodiment.

FIG. 26 is a flowchart that shows monitoring of attachment of the catheter unit with the base unit according to the second embodiment.

FIG. 27 is a diagram that shows an example of the hardware configuration of an information processing apparatus.

### DESCRIPTION OF EMBODIMENTS

Hereinafter, embodiments of the present invention will be described with reference to the drawings. The dimensions, materials, and shapes of components that will be described in the present embodiments, the arrangement of the components, and the like, should be changed as needed depending on the configuration of an apparatus to which the present invention is applied, various conditions, or the like.

### First Embodiment

### Medical System and Medical Device

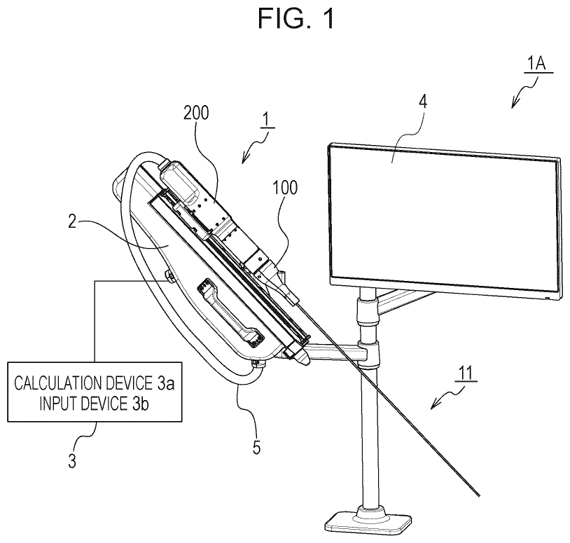

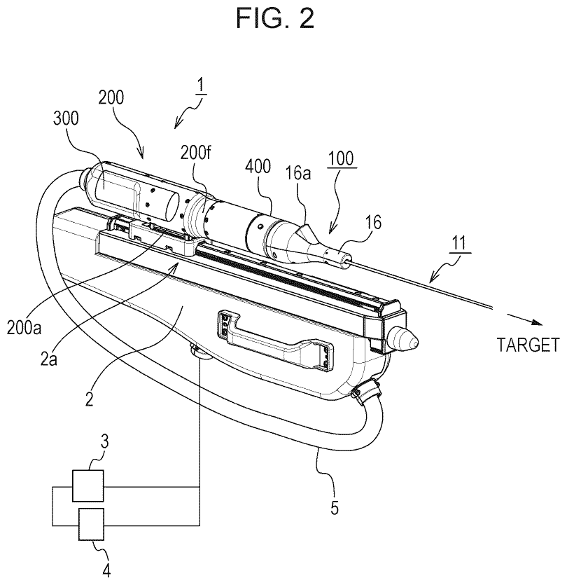

A medical system 1A and a medical device 1 that includes a continuum robot will be described with reference to FIGS. 1 and 2. FIG. 1 is an overall view of the medical system 1A.

FIG. 2 is a perspective view that shows the medical device 1 and a support 2.

The medical system 1A includes the medical device 1, the support 2 that supports the medical device 1, and a controller 3 that controls the medical device 1. In the present embodiment, the medical system 1A includes a monitor 4 serving as a display apparatus.

The medical device 1 includes a catheter unit (bendable unit) 100 including a catheter 11 serving as a bendable body, and a base unit (a drive unit or an attached unit) 200. The catheter unit 100 is configured to be detachably attachable to the base unit 200.

In the present embodiment, a user of the medical system 1A and the medical device 1 is able to do some work, such as observing the inside of a target, collecting various samples from the inside of the target, and treating the inside of the target, by inserting the catheter 11 into the inside of the target. As one of embodiments, a user is able to insert the catheter 11 into the inside of a patient as a target. Specifically, by inserting the catheter 11 into a bronchus via the oral cavity or nasal cavity of a patient, it is possible to do some work, such as observation, collection, and removal of a lung tissue.

The catheter 11 can be used as a guide (sheath) that guides a medical tool for doing the above work. Examples of the medical tool (tool) include an endoscope, a forceps, and an ablation device. The catheter 11 itself may have the functions of the above-described medical tools.

In the present embodiment, the controller 3 is an example of an information processing apparatus to which the present invention is applied and includes a calculation device 3a and an input device 3b. The input device 3b receives a command or input for operating the catheter 11. For example, as will be described later, the input device 3b inputs a detection result of a fixation detection sensor 26b provided in an operating portion 400 to acquire the detection result. The calculation device 3a includes a storage that stores a program and various data for controlling a catheter, a random access memory, and a central processing unit for running the program. The controller 3 may include an output section that outputs a signal for displaying an image on the monitor 4.

As shown in FIG. 2, in the present embodiment, the medical device 1 is electrically connected to the controller 3 via the support 2 and a cable 5 that couples the base unit 200 of the medical device 1 with the support 2. The medical device 1 and the controller 3 may be directly connected by a cable. The medical device 1 and the controller 3 may be wirelessly connected to each other.

The medical device 1 is detachably attached to the support 2 via the base unit 200. More specifically, in the medical device 1, an attachment portion (connecting portion) 200a of the base unit 200 is detachably attached to a movable stage (receiving portion) 2a of the support 2. Even in a state where the attachment portion 200a of the medical device 1 is detached from the movable stage 2a, connection of the medical device 1 with the controller 3 is maintained such that the medical device 1 is controllable by the controller 3. In the present embodiment, even in a state where the attachment portion 200a of the medical device 1 is detached from the movable stage 2a, the medical device 1 and the support 2 are connected by the cable 5.

A user is able to manually move the medical device 1 in a state where the medical device 1 is detached from the support 2 (a state where the medical device 1 is detached from the movable stage 2a) and insert the catheter 11 into the inside of a target.

A user is able to use the medical device 1 in a state where the catheter 11 is inserted in a target and the medical device 1 is attached to the support 2. Specifically, when the movable stage 2a moves in a state where the medical device 1 is attached to the movable stage 2a, the medical device 1 moves. Then, an operation to move the catheter 11 in a direction to be inserted into the target and an operation to move the catheter 11 in a direction to be pulled out from the target are performed. Movement of the movable stage 2a is controlled by the controller 3.

The attachment portion 200a of the base unit 200 includes an unlock switch (not shown) and a detachment switch (not shown). In a state where the attachment portion 200a is attached to the movable stage 2a, a user is able to manually move the medical device 1 along a guide direction of the movable stage 2a while holding down the unlock switch. In other words, the movable stage 2a includes a guide configuration to guide movement of the medical device 1. When the user stops pressing the unlock switch, the medical device 1 is fixed to the movable stage 2a. On the other hand, when the detachment switch is pressed in a state where the attachment portion 200a is attached to the movable stage 2a, the user is able to detach the medical device 1 from the movable stage 2a.

A single switch may be configured to have both the functions of the unlock switch and the detachment switch. When the unlock switch is provided with a mechanism of switching between a pressed down state and a non-pressed down state, a user does not need to hold down the unlock switch when manually sliding the medical device 1.

In a state where the attachment portion 200a is attached to the movable stage 2a and the unlock switch or the detachment switch is not pressed, the medical device 1 is fixed to the movable stage 2a and is moved by the movable stage 2a driven by a motor (not shown).

The medical device 1 includes a wire drive portion (a linear member drive portion, a line drive portion, or a main body drive portion) 300 for actuating the catheter 11. In the present embodiment, the medical device 1 is a robot catheter device that actuates the catheter 11 with the wire drive portion 300 controlled by the controller 3.

The controller 3 can control the wire drive portion 300 and perform an operation to bend the catheter 11. In the present embodiment, the wire drive portion 300 is incorporated in the base unit 200. More specifically, the base unit 200 includes a base housing 200f that accommodates the wire drive portion 300. In other words, the base unit 200 includes the wire drive portion 300. The wire drive portion 300 and the base unit 200 may be collectively referred to as a catheter drive apparatus (a base apparatus or a main body).

In an extending direction of the catheter 11, an end portion at which the distal end of the catheter 11 to be inserted into a target is disposed is referred to as a distal end. In the extending direction of the catheter 11, an opposite side to the distal end is referred to as a proximal end.

The catheter unit 100 has a proximal end cover 16 that covers the proximal end side of the catheter 11. The proximal end cover 16 has a tool hole 16a. A medical tool is allowed to be inserted into the catheter 11 via the tool hole 16a.

As described above, in the present embodiment, the catheter 11 has the function of a guide device for guiding a medical tool to a desired location inside a target.

For example, in a state where an endoscope is inserted in the catheter 11, the catheter 11 is inserted to an intended location inside a target. At this time, at least one of manual operation of a user, movement of the movable stage 2a, and actuation of the catheter 11 with the wire drive portion 300 is used. After the catheter 11 reaches the intended location, the endoscope is pulled out from the catheter 11 via the tool hole 16a. Then, a medical tool is inserted through the tool hole 16a, and some work, such as collecting various samples from the inside of the target and treating the inside of the target, is performed.

As will be described later, the catheter unit 100 is configured to be detachably attachable to the catheter drive apparatus (a base apparatus or a main body), more specifically, the base unit 200. After the medical device 1 is used, a user is able to detach the catheter unit 100 from the base unit 200, attach a new catheter unit 100 to the base unit 200, and use the medical device 1 again.

As shown in FIG. 2, the medical device 1 includes an operating portion 400. In the present embodiment, the operating portion 400 is provided in the catheter unit 100. At the time of attaching the catheter unit 100 to the base unit 200 or detaching the catheter unit 100 from the base unit 200, the operating portion 400 is operated by a user to fix the catheter unit 100 to the base unit 200 or unlock fixing of the catheter unit 100 to the base unit 200.

In the thus configured medical system 1A, for example, by connecting the endoscope inserted in the catheter 11 with the monitor 4, the monitor 4 can display an image taken by the endoscope on the monitor 4. By connecting the monitor 4 with the controller 3, the status of the medical device 1 and information related to control over the medical device 1 can be displayed on the monitor 4. For example, the location of the catheter 11 inside a target or information related to navigation for the catheter 11 inside a target can be displayed on the monitor 4. The monitor 4 and both the controller 3 and the endoscope may be connected by wire or may be connected by wireless. The monitor 4 and the controller 3 may be directly connected via the support 2.

### Catheter

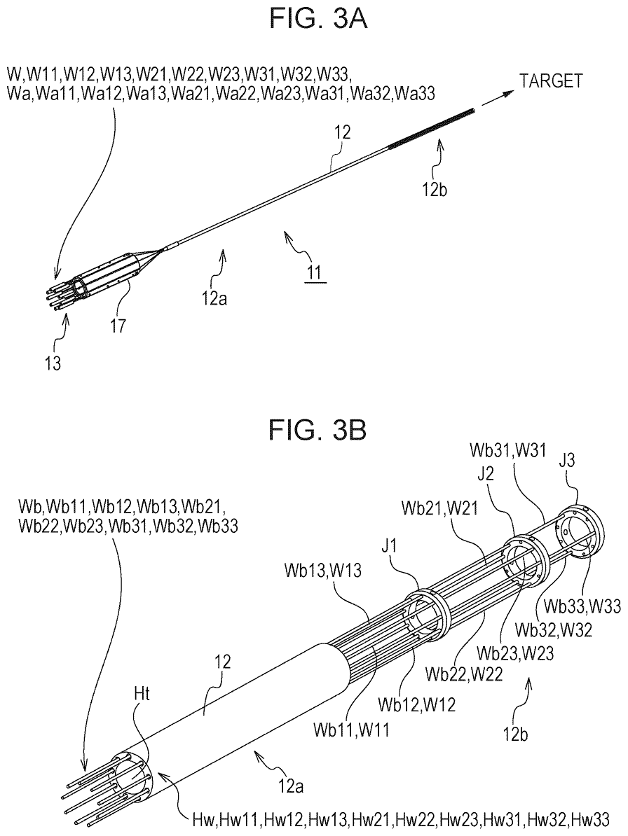

The catheter 11 serving as a bendable body will be described with reference to FIGS. 3A and 3B. FIGS. 3A and 3B are views that illustrate the catheter 11. FIG. 3A is a view that illustrates the whole of the catheter 11. FIG. 3B is an enlarged view of the catheter 11.

The catheter 11 includes a bend portion (a bend body or a catheter main body) 12 and a bend drive portion (catheter drive portion) 13 configured to bend the bend portion 12. The bend drive portion 13 is configured to bend the bend portion 12 upon receiving the driving force of the wire drive portion 300 via a coupling device 21 (described later).

The catheter 11 extends along the direction in which the catheter 11 is inserted into a target. The extending direction (longitudinal direction) of the catheter 11 is the same as an extending direction (longitudinal direction) of the bend portion 12 and an extending direction (longitudinal direction) of each of first to ninth drive wires (W11 to W33) (described later).

The bend drive portion 13 includes the plurality of drive wires (drive lines, linear members, or linear actuators) connected to the bend portion 12. Specifically, the bend drive portion 13 includes the first drive wire W11, the second drive wire W12, the third drive wire W13, the fourth drive wire W21, the fifth drive wire W22, the sixth drive wire W23, the seventh drive wire W31, the eighth drive wire W32, and the ninth drive wire W33.

Each of the first to ninth drive wires (W11 to W33) includes a held portion (a held shaft or a rod) Wa. Specifically, the first drive wire W11 includes the first held portion Wa11. The second drive wire W12 includes the second held portion Wa12. The third drive wire W13 includes the third held portion Wa13. The fourth drive wire W21 includes the fourth held portion Wa21. The fifth drive wire W22 includes the fifth held portion Wa22. The sixth drive wire W23 includes the sixth held portion Wa23. The seventh drive wire W31 includes the seventh held portion Wa31. The eighth drive wire W32 includes the eighth held portion Wa32. The ninth drive wire W33 includes the ninth held portion Wa33.

In the present embodiment, each of the first to ninth held portions (Wa11 to Wa33) has the same shape.

Each of the first to ninth drive wires (W11 to W33) includes a flexible wire body (a line body or a linear body) Wb. Specifically, the first drive wire W11 includes the first wire body Wb11. The second drive wire W12 includes the second wire body Wb12. The third drive wire W13 includes the third wire body Wb13. The fourth drive wire W21 includes the fourth wire body Wb21. The fifth drive wire W22 includes the fifth wire body Wb22. The sixth drive wire W23 includes the sixth wire body Wb23. The seventh drive wire W31 includes the seventh wire body Wb31. The eighth drive wire W32 includes the eighth wire body Wb32. The ninth drive wire W33 includes the ninth wire body Wb33.

In the present embodiment, each of the first to third wire bodies (Wb11 to Wb13) has the same shape. Each of the fourth to sixth wire bodies (Wb21 to Wb23) has the same shape. Each of the seventh to ninth wire bodies (Wb31 to Wb33) has the same shape. In the present embodiment, the first to ninth wire bodies (Wb11 to Wb33) have the same shape.

Each of the first to ninth held portions (Wa11 to Wa33) is fixed to a corresponding one of the first to ninth wire bodies (Wb11 to Wb33) at the proximal end of the corresponding one of the first to ninth wire bodies (Wb11 to Wb33).

The first to ninth drive wires (W11 to W33) are inserted into the bend portion 12 via a wire guide 17 and fixed.

In the present embodiment, the material of each of the first to ninth drive wires (W11 to W33) is a metal. The material of each of the first to ninth drive wires (W11 to W33) may be a resin. The material of each of the first to ninth drive wires (W11 to W33) may include metal and resin.

Of the first to ninth drive wires (W11 to W33), a selected one may be referred to as a drive wire W. In the present embodiment, each of the first to ninth drive wires (W11 to W33) has the same shape except for the length of each of the first to ninth wire bodies (Wb11 to Wb33).

In the present embodiment, the bend portion 12 has flexibility and is a tubular member having a passage Ht for inserting a medical tool.

A wall of the bend portion 12 includes a plurality of wire holes for respectively passing the first to ninth drive wires (W11 to W33). Specifically, the wall of the bend portion 12 includes the first wire hole Hw11, the second wire hole Hw12, the third wire hole Hw13, the fourth wire hole Hw21, the fifth wire hole Hw22, the sixth wire hole Hw23, the seventh wire hole Hw31, the eighth wire hole Hw32, and the ninth wire hole Hw33. The first to ninth wire holes Hw (Hw11 to Hw33) are respectively in correspondence with the first to ninth drive wires (W11 to W33). The numeral suffixed to the sign Hw represents the numeral of a corresponding one of the drive wires. For example, the first drive wire W11 is inserted into the first wire hole Hw11.

Of the first to ninth wire holes (Hw11 to Hw33), a selected one may be referred to as a wire hole Hw. In the present embodiment, each of the first to ninth wire holes (Hw11 to Hw33) has the same shape.

The bend portion 12 has an intermediate region 12a and a bend region 12b. The bend region 12b is disposed at the distal end of the bend portion 12. A first guide ring J1, a second guide ring J2, and a third guide ring J3 are disposed in the bend region 12b. The bend region 12b means a region capable of controlling the degree and direction of bending of the bend portion 12 by moving the first guide ring J1, the second guide ring J2, and the third guide ring J3 with the bend drive portion 13. FIG. 3B is a view drawn by omitting part of the bend portion 12 covering the first to third guide rings (J1 to J3).

In the present embodiment, the bend portion 12 includes a plurality of sub-rings (not shown). In the bend region 12b, the first guide ring J1, the second guide ring J2, and the third guide ring J3 are fixed to the wall of the bend portion 12. In the present embodiment, the sub-rings are respectively disposed on the proximal side with respect to the first guide ring J1, between the first guide ring J1 and the second guide ring J2, and between the second guide ring J2 and the third guide ring J3.

A medical tool is guided to the tip end of the catheter 11 by the passage Ht, the first to third guide rings (J1 to J3), and the plurality of sub-rings.

Each of the first to ninth drive wires (W11 to W33) is fixed to a corresponding one of the first to third guide rings (J1 to J3) through the intermediate region 12a.

Specifically, the first drive wire W11, the second drive wire W12, and the third drive wire W13 extend through the plurality of sub-rings and are fixed to the first guide ring J1. The fourth drive wire W21, the fifth drive wire W22, and the sixth drive wire W23 extend through the first guide ring J1 and the plurality of sub-rings and are fixed to the second guide ring J2. The seventh drive wire W31, the eighth drive wire W32, and the ninth drive wire W33 extend through the first guide ring J1, the second guide ring J2, and the plurality of sub-rings and are fixed to the third guide ring J3.

The medical device 1 is capable of bending the bend portion 12 in a direction that intersects with the extending direction of the catheter 11 by actuating the bend drive portion 13 with the wire drive portion 300. Specifically, by moving each of the first to ninth drive wires (W11 to W33) in the extending direction of the bend portion 12, the bend region 12b of the bend portion 12 is bent in a direction that intersects with the extending direction via the first to third guide rings (J1 to J3).

A user is able to insert the catheter 11 to an intended part inside a target by using at least any one of moving the medical device 1 manually or with the movable stage 2a and bending the bend portion 12.

In the present embodiment, the bend portion 12 is bent by moving the first to third guide rings (J1 to J3) with the first to ninth drive wires (W11 to W33); however, the present invention is not limited to this configuration. Any one or two of the first to third guide rings (J1 to J3) and the drive wires fixed to them may be omitted.

For example, the catheter 11 may have such a configuration that the first to sixth drive wires (W11 to W23) and the first and second guide rings (J1, J2) are omitted and only the seventh to ninth drive wires (W31 to W33) and the third guide ring J3 are provided. Alternatively, the catheter 11 may have such a configuration that the first to third drive wires (W11 to W13) and the first guide ring J1 are omitted and only the fourth to ninth drive wires (W21 to W33) and the second and third guide rings (J2, J3) are provided.

Alternatively, the catheter 11 may have such a configuration that a single guide ring is driven by two drive wires. In this case as well, the number of guide rings may be one or may be more than one.

### Catheter Unit

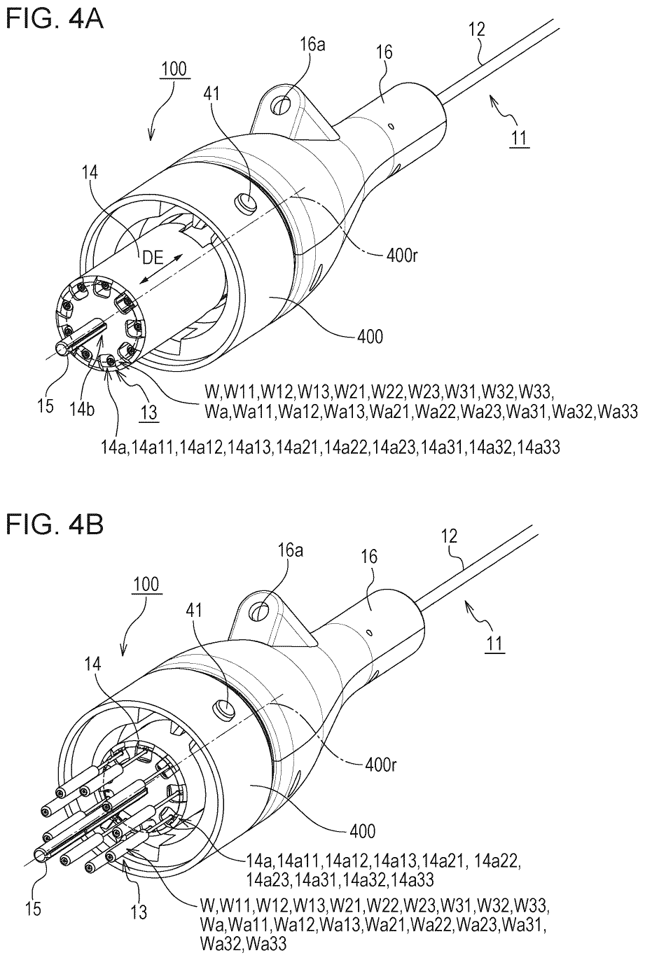

The catheter unit 100 will be described with reference to FIGS. 4A and 4B.

FIGS. 4A and 4B are views that illustrate the catheter unit 100. FIG. 4A is a view that illustrates the catheter unit 100 in a state where a wire cover 14 (described later) is at a cover position. FIG. 4B is a view that illustrates the catheter unit 100 in a state where the wire cover 14 (described later) is at a retracted position.

The catheter unit 100 includes the catheter 11 including the bend portion 12 and the bend drive portion 13, and a proximal end cover 16 that supports the proximal end of the catheter 11. The catheter unit 100 includes the cover (wire cover) 14 for covering and protecting the first to ninth drive wires (W11 to W33) serving as the plurality of drive wires.

The catheter unit 100 is detachably attachable to the base unit 200 along an attaching and detaching direction DE. The direction in which the catheter unit 100 is attached to the base unit 200 and the direction in which the catheter unit 100 is detached from the base unit 200 are parallel to the attaching and detaching direction DE.

The proximal end cover (a frame, a bend portion housing, or a catheter housing) 16 is a cover covering part of the catheter 11. The proximal end cover 16 has the tool hole 16a for inserting a medical tool into the passage Ht of the bend portion 12.

The wire cover 14 has a plurality of wire cover holes (cover holes) for respectively passing the first to ninth drive wires (W11 to W33). The wire cover 14 has the first wire cover hole 14a11, the second wire cover hole 14a12, the third wire cover hole 14a13, the fourth wire cover hole 14a21, the fifth wire cover hole 14a22, the sixth wire cover hole 14a23, the seventh wire cover hole 14a31, the eighth wire cover hole 14a32, and the ninth wire cover hole 14a33. The first to ninth wire cover holes (14a11 to 14a33) are respectively in correspondence with the first to ninth drive wires (W11 to W33). The numeral suffixed to the sign 14a represents the numeral of a corresponding one of the drive wires. For example, the first drive wire W11 is inserted into the first wire cover hole 14a11.

Of the first to ninth wire cover holes (14a11 to 14a33), a selected one may be referred to as a wire cover hole 14a. In the present embodiment, each of the first to ninth wire cover holes (14a11 to 14a33) has the same shape.

The wire cover 14 can be moved to a cover position (see FIG. 4A) where the first to ninth drive wires (W11 to W33) are covered and a retracted position (see FIG. 4B) retracted from the cover position. The retracted position may also be referred to as an exposed position where the first to ninth drive wires (W11 to W33) are exposed.

Before the catheter unit 100 is attached to the base unit 200, the wire cover 14 is located at the cover position. When the catheter unit 100 is attached to the base unit 200, the wire cover 14 moves from the cover position to the retracted position along the attaching and detaching direction DE.

In the present embodiment, after the wire cover 14 is moved from the cover position to the retracted position, the wire cover 14 is retained at the retracted position. Therefore, even when the catheter unit 100 is attached to the base unit 200 and then the catheter unit 100 is detached from the base unit 200, the wire cover 14 is retained at the retracted position.

However, the wire cover 14 may be configured to, after the wire cover 14 is moved from the cover position to the retracted position, return to the cover position. For example, the catheter unit 100 may include an urging member that urges the wire cover 14 from the retracted position toward the cover position. In this case, when the catheter unit 100 is detached from the base unit 200 after the catheter unit 100 is attached to the base unit 200, the wire cover 14 is moved from the retracted position to the cover position.

When the wire cover 14 is at the retracted position, the first to ninth held portions (Wa11 to Wa33) of the first to ninth drive wires (W11 to W33) protrude with respect to the wire cover 14. As a result, coupling of the bend drive portion 13 with the coupling device 21 (described later) is allowed. When the wire cover 14 is at the retracted position, the first to ninth held portions (Wa11 to Wa33) of the first to ninth drive wires (W11 to W33) protrude from the first to ninth wire cover holes (14a11 to 14a33). More specifically, the first to ninth held portions (Wa11 to Wa33) protrude from the first to ninth wire cover holes (14a11 to 14a33) in an attachment direction Da (described later).

As shown in FIG. 4B, the first to ninth drive wires (W11 to W33) are arranged along a circle (imaginary circle) having a predetermined radius.

In the present embodiment, the catheter unit 100 includes a key shaft (a key or a catheter-side key) 15. In the present embodiment, the key shaft 15 extends in the attaching and detaching direction DE. The wire cover 14 has a shaft hole 14b through which the key shaft 15 extends.

The key shaft 15 is engageable with a key receiving portion 22 (described later). When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

In the present embodiment, when viewed in the attaching and detaching direction DE, the first to ninth drive wires (W11 to W33) are disposed outside the key shaft 15 so as to surround the key shaft 15. In other words, the key shaft 15 is disposed inside the circle (imaginary circle) along which the first to ninth drive wires (W11 to W33) are arranged. Therefore, the key shaft 15 and the first to ninth drive wires (W11 to W33) can be disposed in a space-saving manner.

In the present embodiment, the catheter unit 100 includes the operating portion 400. The operating portion 400 is configured to be movable (rotatable) with respect to the proximal end cover 16 and the bend drive portion 13.

The operating portion 400 is rotatable around a rotation axis 400r. The rotation axis 400r of the operating portion 400 extends in the attaching and detaching direction DE.

In a state where the catheter unit 100 is attached to the base unit 200, the operating portion 400 is configured to be movable (rotatable) with respect to the base unit 200. More specifically, the operating portion 400 is configured to be movable (rotatable) with respect to the base housing 200f, the wire drive portion 300, and the coupling device 21 (described later).

### Base Unit

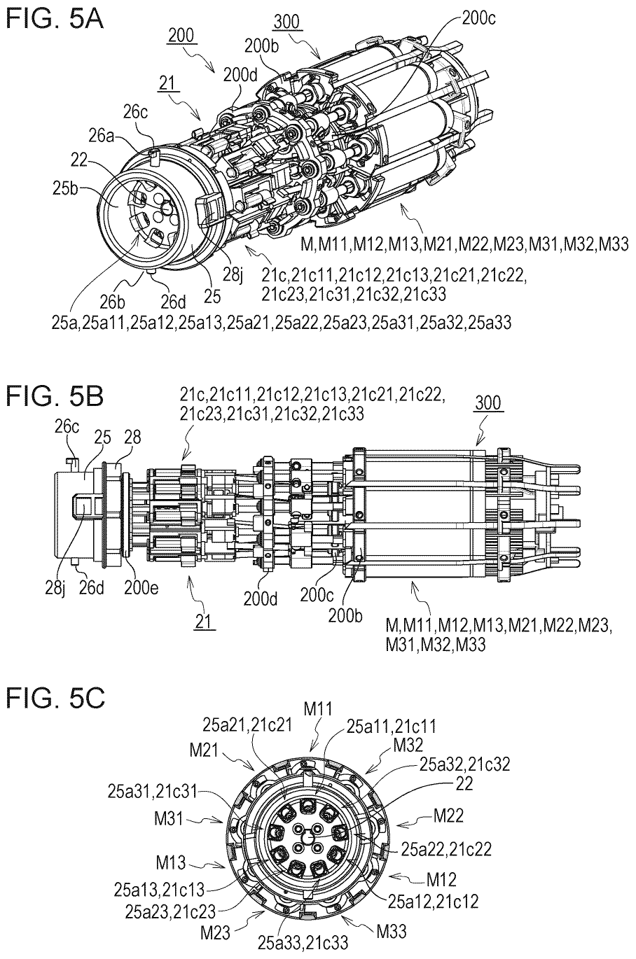

The base unit 200 and the wire drive portion 300 will be described with reference to FIGS. 5A, 5B, and 5C.

FIGS. 5A, 5B, and 5C are views that illustrate the base unit 200 and the wire drive portion 300. FIG. 5A is a perspective view that shows the internal structure of the base unit 200. FIG. 5B is a side view that shows the internal structure of the base unit 200. FIG. 5C is a view of the base unit 200 when viewed along the attaching and detaching direction DE.

As described above, the medical device 1 includes the base unit 200 and the wire drive portion 300. In the present embodiment, the wire drive portion 300 is accommodated in the base housing 200f and is provided inside the base unit 200. In other words, the base unit 200 includes the wire drive portion 300.

The wire drive portion 300 includes a plurality of driving sources (motors). In the present embodiment, the wire drive portion 300 includes the first driving source M11, the second driving source M12, the third driving source M13, the fourth driving source M21, the fifth driving source M22, the sixth driving source M23, the seventh driving source M31, the eighth driving source M32, and the ninth driving source M33.

Of the first to ninth driving sources (M11 to M33), a selected one may be referred to as a driving source M. In the present embodiment, each of the first to ninth driving sources (M11 to M33) has the same configuration.

The base unit 200 includes the coupling device 21. The coupling device 21 is accommodated in the base housing 200f. The coupling device 21 is connected to the wire drive portion 300. The coupling device 21 includes a plurality of coupling portions. In the present embodiment, the coupling device 21 includes the first coupling portion 21c11, the second coupling portion 21c12, the third coupling portion 21c13, the fourth coupling portion 21c21, the fifth coupling portion 21c22, the sixth coupling portion 21c23, the seventh coupling portion 21c31, the eighth coupling portion 21c32, and the ninth coupling portion 21c33.

Of the first to ninth coupling portions (21c11 to 21c33), a selected one may be referred to as a coupling portion 21c. In the present embodiment, each of the first to ninth coupling portions (21c11 to 21c33) has the same configuration.

Each of the plurality of coupling portions is connected to a corresponding one of the plurality of driving sources and is driven by the corresponding one of the plurality of driving sources. Specifically, the first coupling portion 21c11 is connected to the first driving source M11 and is driven by the first driving source M11. The second coupling portion 21c12 is connected to the second driving source M12 and is driven by the second driving source M12. The third coupling portion 21c13 is connected to the third driving source M13 and is driven by the third driving source M13. The fourth coupling portion 21c21 is connected to the fourth driving source M21 and is driven by the fourth driving source M21. The fifth coupling portion 21c22 is connected to the fifth driving source M22 and is driven by the fifth driving source M22. The sixth coupling portion 21c23 is connected to the sixth driving source M23 and is driven by the sixth driving source M23. The seventh coupling portion 21c31 is connected to the seventh driving source M31 and is driven by the seventh driving source M31. The eighth coupling portion 21c32 is connected to the eighth driving source M32 and is driven by the eighth driving source M32. The ninth coupling portion 21c33 is connected to the ninth driving source M33 and is driven by the ninth driving source M33.

As will be described later, the bend drive portion 13 including the first to ninth drive wires (W11 to W33) is coupled to the coupling device 21. The bend drive portion 13 receives the driving force of the wire drive portion 300 via the coupling device 21 to bend the bend portion 12.

The drive wire W is coupled to the coupling portion 21c via the held portion Wa. Each of the plurality of drive wires is coupled to a corresponding one of the plurality of coupling portions.

Specifically, the first held portion Wa11 of the first drive wire W11 is coupled to the first coupling portion 21cl1. The second held portion Wa12 of the second drive wire W12 is coupled to the second coupling portion 21c12. The third held portion Wa13 of the third drive wire W13 is coupled to the third coupling portion 21c13. The fourth held portion Wa21 of the fourth drive wire W21 is coupled to the fourth coupling portion 21c21. The fifth held portion Wa22 of the fifth drive wire W22 is coupled to the fifth coupling portion 21c22. The sixth held portion Wa23 of the sixth drive wire W23 is coupled to the sixth coupling portion 21c23. The seventh held portion Wa31 of the seventh drive wire W31 is coupled to the seventh coupling portion 21c31. The eighth held portion Wa32 of the eighth drive wire W32 is coupled to the eighth coupling portion 21c32. The ninth held portion Wa33 of the ninth drive wire W33 is coupled to the ninth coupling portion 21c33.

The base unit 200 includes a base frame 25. The base frame 25 has a plurality of insertion holes for respectively passing the first to ninth drive wires (W11 to W33). The base frame 25 has the first insertion hole 25a11, the second insertion hole 25a12, the third insertion hole 25a13, the fourth insertion hole 25a21, the fifth insertion hole 25a22, the sixth insertion hole 25a23, the seventh insertion hole 25a31, the eighth insertion hole 25a32, and the ninth insertion hole 25a33. The first to ninth insertion holes (25a11 to 25a33) are respectively in correspondence with the first to ninth drive wires (W11 to W33). The numeral suffixed to the sign 25a represents the numeral of a corresponding one of the drive wires. For example, the first drive wire W11 is inserted into the first insertion hole 25a11.

Of the first to ninth insertion holes (25a11 to 25a33), a selected one may be referred to as an insertion hole 25a. In the present embodiment, each of the first to ninth insertion holes (25a11 to 25a33) has the same shape.

The base frame 25 has an attachment opening 25b into which the wire cover 14 is inserted. The first to ninth insertion holes (25a11 to 25a33) are disposed at the bottom portion of the attachment opening 25b.

In addition, the base unit 200 includes a motor frame 200b, a first bearing frame 200c, a second bearing frame 200d, and a third bearing frame 200e. The motor frame 200b, the first bearing frame 200c, the second bearing frame 200d, and the third bearing frame 200e are coupled to one another.

The base frame 25 has the key receiving portion (a key hole, a base-side key, or a main body-side key) 22 for receiving the key shaft 15. When the key shaft 15 and the key receiving portion 22 are engaged with each other, the catheter unit 100 is attached to the base unit 200 in proper phase.

When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

As a result, each of the first to ninth drive wires (W11 to W33) is engaged with a corresponding one of the first to ninth insertion holes (25a11 to 25a33) and a corresponding one of the first to ninth coupling portions (21c11 to 21c33). In other words, engagement of the drive wire W with a non-corresponding one of the insertion holes 25a and a non-corresponding one of the coupling portions 21c is prevented.

A user is able to properly couple each of the first to ninth drive wires (W11 to W33) with a corresponding one of the first to ninth coupling portions (21c11 to 21c33) by engaging the key shaft 15 with the key receiving portion 22. Therefore, a user is able to easily attach the catheter unit 100 to the base unit 200.

In the present embodiment, the key shaft 15 has a protruding portion that protrudes in a direction to intersect with the attaching and detaching direction DE, and the key receiving portion 22 has a recess portion into which the protruding portion is inserted. A position in which the protruding portion and the recess portion are engaged with each other in the circumferential direction is a position in which each of the drive wires W is engaged with a corresponding one of the insertion holes 25a and a corresponding one of the coupling portions 21c.

The key shaft 15 may be disposed in any one of the base unit 200 and the catheter unit 100, and the key receiving portion 22 may be disposed in the other. For example, the key shaft 15 may be disposed at the base unit 200 side, and the key receiving portion 22 may be disposed at the catheter unit 100 side.

The base unit 200 has a joint 28 that includes a joint engagement portion 28j (described later).

The base frame 25 has two connection shafts 26c, 26d (described later).

Coupling of Motor with Drive Wire

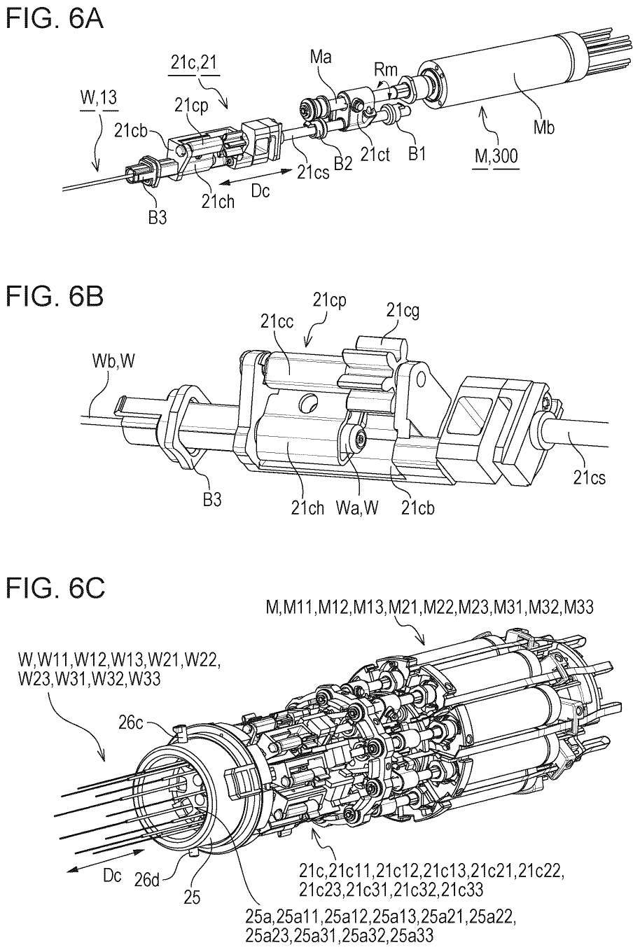

Coupling among the wire drive portion 300, the coupling device 21, and the bend drive portion 13 will be described with reference to FIGS. 6A, 6B, and 6C.

FIGS. 6A, 6B, and 6C are views that illustrate the wire drive portion 300, the coupling device 21, and the bend drive portion 13. FIG. 6A is a perspective view of the driving source M, the coupling portion 21c, and the drive wire W. FIG. 6B is an enlarged view of the coupling portion 21c and the drive wire W. FIG. 6C is a perspective view that shows coupling among the wire drive portion 300, the coupling device 21, and the bend drive portion 13.

In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth driving sources (M11 to M33) is the same. Therefore, in the following description, the configuration in which the drive wire W, the coupling portion 21c, and the driving source M are connected will be described by using the one drive wire w, the one coupling portion 21c, and the one driving source M.

As shown in FIG. 6A, the driving source M includes an output shaft Ma that is a motor shaft and a motor main body Mb that rotates the output shaft Ma in a rotation direction Rm. A spiral groove is provided on the surface of the output shaft Ma. The output shaft Ma has a so-called screw shape. The motor main body Mb is fixed to the motor frame 200b.

The coupling portion 21c has a tractor 21ct connected to the output shaft Ma and a tractor support shaft 21cs that supports the tractor 21ct. The tractor support shaft 21cs is connected to a coupling base 21cb.

The coupling portion 21c includes a leaf spring 21ch serving as a holding portion for holding the held portion Wa of the drive wire W. The drive wire W is engaged with the coupling portion 21c through the insertion hole 25a. More specifically, the held portion Wa is engaged with the leaf spring 21ch. As will be described later, the leaf spring 21ch can be placed in a state where the held portion Wa is clamped and fixed (fixed state) and a state where the held portion Wa is released (released state).

The coupling portion 21c includes a pressing member 21cp. As shown in FIG. 6B, the pressing member 21cp has a gear portion 21cg meshed with an internal gear 29 (described later) and a cam 21cc serving as a pressing portion for pressing the leaf spring 21ch.

As will be described later, the cam 21cc can move with respect to the leaf spring 21ch. When the cam 21cc moves, the leaf spring 21ch is switched between the fixed state and the released state.

The coupling portion 21c is supported by a first bearing B1, a second bearing B2, and a third bearing B3. The first bearing B1 is supported by the first bearing frame 200c of the base unit 200. The second bearing B2 is supported by the second bearing frame 200d of the base unit 200. The third bearing B3 is supported by the third bearing frame 200e of the base unit 200. Therefore, when the output shaft Ma rotates in the rotation direction Rm, rotation of the coupling portion 21c around the output shaft Ma is restricted. The first bearing B1, the second bearing B2, and the third bearing B3 are provided for each of the first to ninth coupling portions (21c11 to 21c33).

Since rotation of the coupling portion 21c around the output shaft Ma is restricted, when the output shaft Ma rotates, a force along the rotational axis direction of the output shaft Ma is applied to the tractor 21ct by the spiral groove of the output shaft Ma. As a result, the coupling portion 21c moves along the rotational axis direction of the output shaft Ma (Dc direction). When the coupling portion 21c moves, the drive wire W moves, and the bend portion 12 bends.

In other words, the output shaft Ma and the tractor 21ct make up a so-called feed screw that converts rotational motion transmitted from the driving source M to linear motion by a screw. In the present embodiment, the output shaft Ma and the tractor 21ct make up a slide screw. Alternatively, the output shaft Ma and the tractor 21ct may make up a ball screw.

As shown in FIG. 6C, by attaching the catheter unit 100 to the base unit 200, each of the first to ninth drive wires (W11 to W33) and a corresponding one of the first to ninth coupling portions (21c11 to 21c33) are coupled to each other.

The controller 3 is capable of controlling the first to ninth driving sources (M11 to M33) independently of each other. In other words, a selected one driving source of the first to ninth driving sources (M11 to M33) is allowed to independently operate or stop regardless of whether the other driving sources are stopped. In other words, the controller 3 is capable of controlling each of the first to ninth drive wires (W11 to W33) independently of one another. As a result, each of the first to third guide rings (J1 to J3) is controlled independently of one another, and the bend region 12b of the bend portion 12 is allowed to bend in a selected direction.

### Attachment of Catheter Unit

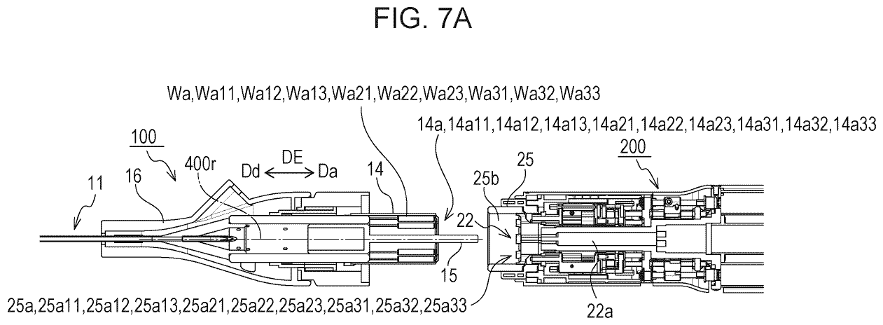

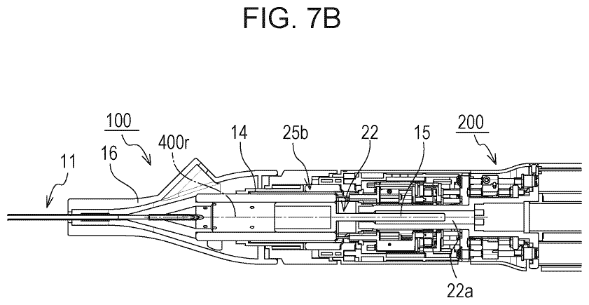

An operation to attach the catheter unit 100 to the base unit 200 will be described with reference to FIGS. 7A and 7B.

FIGS. 7A and 7B are views that illustrate attachment of the catheter unit 100. FIG. 7A is a view before the catheter unit 100 is attached to the base unit 200. FIG. 7B is a view after the catheter unit 100 is attached to the base unit 200.

In the present embodiment, the attaching and detaching direction DE of the catheter unit 100 is the same as the direction of the rotation axis 400r of the operating portion 400. In the attaching and detaching direction DE, the direction in which the catheter unit 100 is attached to the base unit 200 is referred to as the attachment direction Da. In the attaching and detaching direction DE, the direction in which the catheter unit 100 is detached from the base unit 200 (a direction opposite to the attachment direction Da) is referred to as a detachment direction Dd.

As shown in FIG. 7A, in a state before the catheter unit 100 is attached to the base unit 200, the wire cover 14 is placed at the cover position. At this time, the wire cover 14 covers the first to ninth drive wires (W11 to W33) such that the first to ninth held portions (Wa11 to Wa33) do not protrude from the first to ninth wire cover holes (14a11 to 14a33) of the wire cover 14. Therefore, in a state before the catheter unit 100 is attached to the base unit 200, the first to ninth drive wires (W11 to W33) are protected.

When the catheter unit 100 is attached to the base unit 200, the key shaft 15 is engaged with the key receiving portion 22. The key shaft 15 protrudes from the wire cover 14. A key hole 22a forming the key receiving portion 22 extends in the attachment direction Da of the key shaft 15. In the present embodiment, in a state where the key shaft 15 has reached the entrance of the key receiving portion 22, the wire cover 14 is not engaged with the attachment opening 25b. In other words, when the phase of the catheter unit 100 with respect to the base unit 200 is a phase in which the key shaft 15 and the key receiving portion 22 are not engaged with each other, the wire cover 14 is not engaged with the attachment opening 25b, and the state where the wire cover 14 is at the cover position is maintained. Therefore, even when the catheter unit 100 is moved such that the key shaft 15 and the key receiving portion 22 are engaged with each other, the first to ninth drive wires (W11 to W33) are protected.

When the key shaft 15 and the key receiving portion 22 are engaged with each other and the catheter unit 100 is moved in the attachment direction Da with respect to the base unit 200, the catheter unit 100 is attached to the base unit 200. When the catheter unit 100 is attached to the base unit 200, the wire cover 14 is moved to the retracted position. In the present embodiment, the wire cover 14 contacts with the base frame 25 to move from the cover position to the retracted position (see FIG. 7B).

More specifically, when the catheter unit 100 is attached, the wire cover 14 contacts with the base frame 25 to stop. In this state, by moving the catheter unit 100 in the attachment direction Da, the wire cover 14 relatively moves with respect to a part other than the wire cover 14 in the catheter unit 100. As a result, the wire cover 14 moves from the cover position to the retracted position.

The wire cover 14 moves from the cover position to the retracted position, while the held portion Wa of the drive wire W protrudes from the wire cover hole 14a of the wire cover 14 and is inserted into the insertion hole 25a. Then, the held portion Wa is engaged with the leaf spring 21ch of the coupling portion 21c (see FIG. 6B).

Here, more specifically, attachment of the catheter unit 100 with the base unit 200 includes a temporarily attached state and an attachment completed state.

In other words, when the key shaft 15 and the key receiving portion 22 are engaged with each other and the catheter unit 100 is moved in the attachment direction Da with respect to the base unit 200, the catheter unit 100 is attached to the base unit 200. This state is referred to as a temporarily attached state. In the temporarily attached state, the catheter unit 100 can be detached by moving the catheter unit 100 in the detachment direction Dd with respect to the base unit 200. As will be described later, in the temporarily attached state, fixing of the drive wire W with the coupling portion 21c is unlocked.

By operating the operating portion 400 to move to the fixed position (described later) in the temporarily attached state, the state becomes the attachment completed state, with the result that detachment of the catheter unit 100 from the base unit 200 is prevented. In the attachment completed state, the bend drive portion 13 is fixed to the coupling device 21, and the bend drive portion 13 is coupled to the wire drive portion 300 via the coupling device 21.

### Fixing of Bend Drive Portion and Unlocking of Fixing

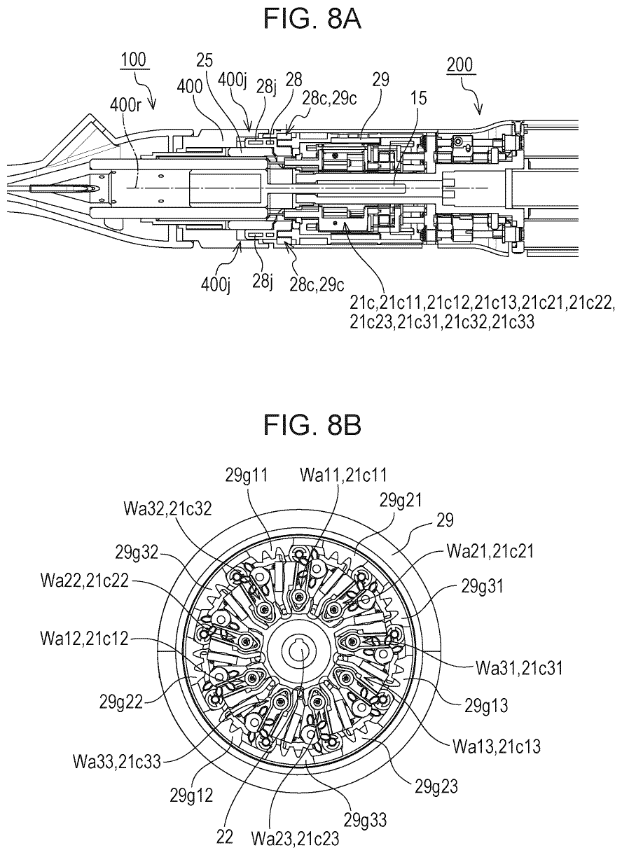

A configuration for fixing the bend drive portion 10 to the coupling device 21 and a configuration for unlocking fixing of the bend drive portion 13 with the coupling device 21 will be described with reference to FIGS. 8A, 8B, 9, 10, 11, 12, 13, and 14.

FIGS. 8A and 8B are views that illustrate coupling of the catheter unit 100 with the base unit 200. FIG. 8A is a sectional view of the catheter unit 100 and the base unit 200. FIG. 8A is a sectional view of the catheter unit 100 and the base unit 200, taken along the rotation axis 400r. FIG. 8B is a sectional view of the base unit 200. FIG. 8B is a sectional view of the base unit 200, taken in a direction orthogonal to the rotation axis 400r at a part of the coupling portion 21c.

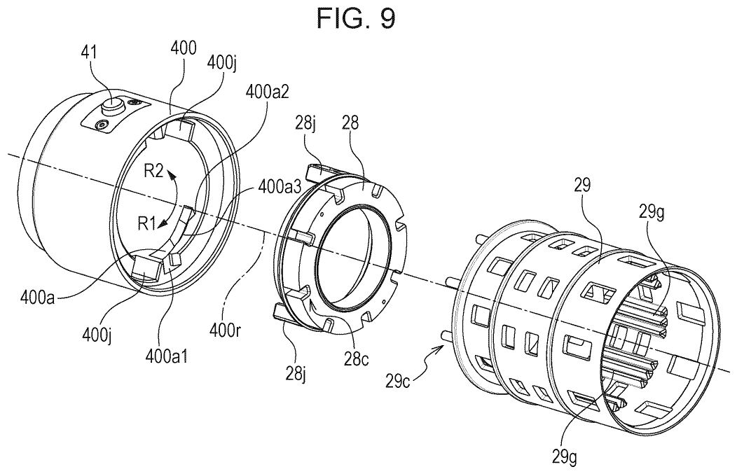

FIG. 9 is an exploded view that illustrates coupling of the catheter unit 100 with the base unit 200.

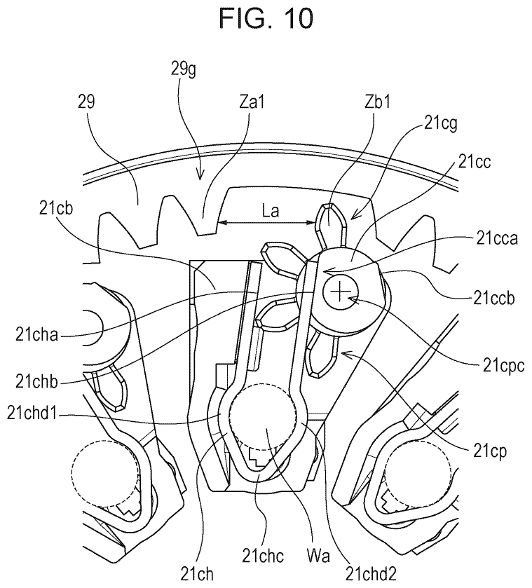

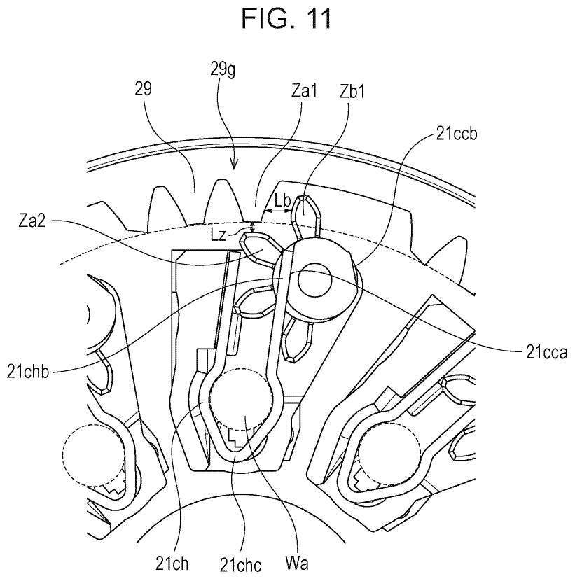

FIGS. 10, 11, 12, 13, and 14 are views that illustrate fixing of the drive wire W with the coupling portion 21c.

As shown in FIGS. 8A and 9, the base unit 200 includes the joint (an intermediate member or a second transmission member) 28, and the internal gear 29 serving as a movable gear (an interlocking gear, a transmission member, or a first transmission member) that is in interlocking with the operating portion 400 via the joint 28.

The joint 28 has a plurality of transmitting portions 28c. The internal gear 29 has a plurality of transmitted portions 29c. The plurality of transmitting portions 28c is engaged with the plurality of transmitted portions 29c, and, when the joint 28 rotates, rotation of the joint 28 is transmitted to the internal gear 29.

When the catheter unit 100 is attached to the base unit 200, the engagement portion 400j provided in the operating portion 400 is engaged with the joint engagement portion 28j of the joint 28. When the operating portion 400 rotates, rotation of the operating portion 400 is transmitted to the joint 28. The operating portion 400, the joint 28, and the internal gear 29 rotate in the same direction.

The internal gear 29 has a plurality of tooth portions for switching between a state where each of the first to ninth coupling portions (21c11 to 21c33) fixes a corresponding one of the first to ninth drive wires (W11 to W33) and a state where each of the first to ninth coupling portions (21c11 to 21c33) releases a corresponding one of the first to ninth drive wires (W11 to W33). Each of the plurality of tooth portions (working portions or gear switching portions) of the internal gear 29 is engaged with the gear portion 21cg of the pressing member 21cp of each of the first to ninth coupling portions (21c11 to 21c33).

Specifically, in the present embodiment, the internal gear 29 has the first tooth portion 29g11, the second tooth portion 29g12, the third tooth portion 29g13, the fourth tooth portion 29g21, the fifth tooth portion 29g22, the sixth tooth portion 29g23, the seventh tooth portion 29g31, the eighth tooth portion 29g32, and the ninth tooth portion 29g33. The first to ninth tooth portions (29g11 to 29g33) are formed with a gap from each other.

The first tooth portion 29g11 meshes with the gear portion 21cg of the first coupling portion 21c11. The second tooth portion 29g12 meshes with the gear portion 21cg of the second coupling portion 21c12. The third tooth portion 29g13 meshes with the gear portion 21cg of the third coupling portion 21c13. The fourth tooth portion 29g21 meshes with the gear portion 21cg of the fourth coupling portion 21c21. The fifth tooth portion 29g22 meshes with the gear portion 21cg of the fifth coupling portion 21c22. The sixth tooth portion 29g23 meshes with the gear portion 21cg of the sixth coupling portion 21c23. The seventh tooth portion 29g31 meshes with the gear portion 21cg of the seventh coupling portion 21c31. The eighth tooth portion 29g32 meshes with the gear portion 21cg of the eighth coupling portion 21c32. The ninth tooth portion 29g33 meshes with the gear portion 21cg of the ninth coupling portion 21c33.

Of the first to ninth tooth portions (29g11 to 29g33), a selected one may be referred to as a tooth portion 29g. In the present embodiment, each of the first to ninth tooth portions (29g11 to 29g33) has the same configuration.

In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth tooth portions (29g11 to 29g33) is the same. Therefore, in the following description, the configuration in which the drive wire W, the coupling portion 21c, and the tooth portion 29g are connected will be described by using the one drive wire W, the one coupling portion 21c, and the one tooth portion 29g.

In each of the first to ninth coupling portions (21c11 to 21c33), when the gear portion 21cg is moved by the internal gear 29, the pressing member 21cp rotates, and the cam 21cc moves to a pressing position or to a retracted position retracted from the pressing position.

By rotating the operating portion 400, the internal gear 29 rotates. When the internal gear 29 rotates, each of the first to ninth coupling portions (21c11 to 21c33) operates. In this way, with an operation to rotate the one operating portion 400, the first to ninth coupling portions (21c11 to 21c33) are actuated.

The operating portion 400 is allowed to move to a fixed position and a detachment position in a state where the catheter unit 100 is attached to the base unit 200. As will be described later, the operating portion 400 is allowed to move to the unlock position in a state where the catheter unit 100 is attached to the base unit 200. In the rotation direction of the operating portion 400, the unlock position is located between the fixed position and the detachment position. In a state where the operating portion 400 is at the detachment position, the catheter unit 100 is attached to the base unit 200 (temporarily attached state).

In the temporarily attached state, the drive wire W is not fixed (locked) to the coupling portion 21c. This state is referred to as an unlocked state of the coupling portion 21c. A state where the drive wire W is fixed (locked) to the coupling portion 21c is referred to as a locked state of the coupling portion 21c.

An operation to fix the drive wire W to the coupling portion 21c will be described with reference to FIGS. 10, 11, 12, 13, and 14.

In the temporarily attached state, that is, in a state after the catheter unit 100 is attached to the base unit 200 and before the operating portion 400 is operated, the catheter unit 100 is allowed to be detached from the base unit 200. Hereinafter, a state where the catheter unit 100 is allowed to be detached from the base unit 200 is referred to as a detachable state.

FIG. 10 is a view that shows a state of the internal gear 29 and the coupling portion 21c in the detachable state. FIG. 10 is a view that shows a state of the internal gear 29 and the coupling portion 21c in a state where the operating portion 400 is at the detachment position.

The leaf spring 21ch of the coupling portion 21c has a fixed portion 21cha fixed to the coupling base 21cb, and a pressed portion 21chb that contacts with the cam 21cc of the pressing member 21cp. The leaf spring 21ch has a first part 21chd1 and a second part 21chd2. When the catheter unit 100 is attached to the base unit 200, the held portion Wa is inserted between the first part 21chd1 and the second part 21chd2.

The cam 21cc has a holding surface 21cca and a pressing surface 21ccb. In a radial direction of rotation of the pressing member 21cp, the holding surface 21cca is disposed at a position closer to a rotation center 21cpc of the pressing member 21cp than the pressing surface 21ccb.

As shown in FIG. 10, in the detachable state (a state where the operating portion 400 is at the detachment position), the leaf spring 21ch is held at a position at which the pressed portion 21chb is in contact with the holding surface 21cca. A tooth Za1 of the internal gear 29 and a tooth Zb1 of the gear portion 21cg are stopped in a state where there is a clearance La therebetween.

In the rotation direction of the operating portion 400, a direction in which the operating portion 400 heads from the detachment position for the unlock position and the fixed position is referred to as a lock direction (fixing direction), and a direction in which the operating portion 400 heads from the fixed position for the unlock position and the detachment position is referred to as an unlock direction. The operating portion 400 rotates in the unlock direction from the unlock position and moves to the detachment position. The operating portion 400 rotates in the lock direction from the unlock position to move to the fixed position.

In the temporarily attached state, the coupling portion 21c is in an unlocked state, and fixing of the drive wire W with the coupling portion 21c is unlocked.

When the coupling portion 21c is in the unlocked state, the cam 21cc is placed at the retracted position retracted from the pressing position (described later). At this time, fixing of the held portion Wa with the leaf spring 21ch is unlocked. A force that the first part 21chd1 and the second part 21chd2 fasten the held portion Wa when the coupling portion 21c is in the unlocked state is less than a force that the first part 21chd1 and the second part 21chd2 fasten the held portion Wa when the coupling portion 21c is in the locked state.

When the catheter unit is moved in the detachment direction Dd with respect to the base unit 200 while the coupling portion 21c is in the unlocked state, the held portion Wa can be pulled out from between the first part 21chd1 and the second part 21chd2.

It is desirable that no force that the first part 21chd1 and the second part 21chd2 fasten the held portion Wa be generated (a state where the magnitude is zero) when the coupling portion 21c is in the unlocked state. It is desirable that a clearance be formed between the held portion Wa and at least any one of the first part 21chd1 and the second part 21chd2 when the coupling portion 21c is in the unlocked state.

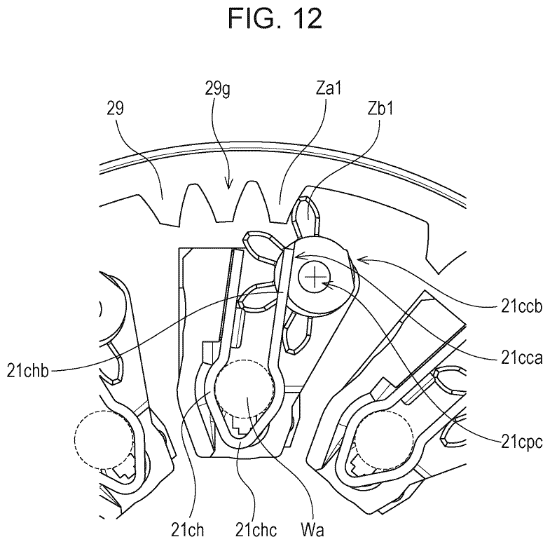

FIG. 11 is a view that shows a state of the internal gear 29 and the coupling portion 21c when the operating portion 400 is rotated in the lock direction from the detachment position. FIG. 11 is a view that shows a state of the internal gear 29 and the coupling portion 21c in a state where the operating portion 400 is at the unlock position.

When the operating portion 400 is rotated in the lock direction in a state where the operating portion 400 is at the detachment position (FIG. 10), the internal gear 29 rotates in the clockwise direction. Then, the operating portion 400 is at the unlock position.

Even when the operating portion 400 is rotated, the key shaft 15 and the key receiving portion 22 are engaged with each other, so rotation of the whole (except the operating portion 400) of the catheter unit 100 with respect to the base unit 200 is restricted. In other words, the operating portion 400 is rotatable with respect to the whole (except the operating portion 400) of the catheter unit 100 and the base unit 200 in a state where the whole (except the operating portion 400) of the catheter unit 100 and the base unit 200 are stopped.

When the internal gear 29 rotates in the clockwise direction, the clearance between the tooth Za1 of the internal gear 29 and the tooth Zb1 of the gear portion 21cg reduces from the clearance La to a clearance Lb.

A tooth Zb2 of the gear portion 21cg is disposed at a position spaced a clearance Lz apart from a tip circle (dashed line) of the tooth portion 29g of the internal gear 29. Therefore, the internal gear 29 is rotatable without interference with the tooth Zb2. On the other hand, the coupling portion 21c is maintained in the same state (unlocked state) as the state shown in FIG. 10.

When the operating portion 400 is further rotated from the state shown in FIG. 11 in the lock direction, the internal gear 29 is further rotated in the clockwise direction. FIG. 12 shows a state of the internal gear 29 and the coupling portion 21c at that time.

FIG. 12 is a view that shows a state of the internal gear 29 and the coupling portion 21c when the operating portion 400 is rotated in the lock direction from the unlock position.

As shown in FIG. 12, when the operating portion 400 is rotated in the lock direction from the unlock position, the tooth Za1 of the internal gear 29 and the tooth Zb1 of the gear portion 21cg contact with each other. On the other hand, the coupling portion 21c is maintained in the unlocked state that is the same state as the state shown in FIGS. 10 and 11.

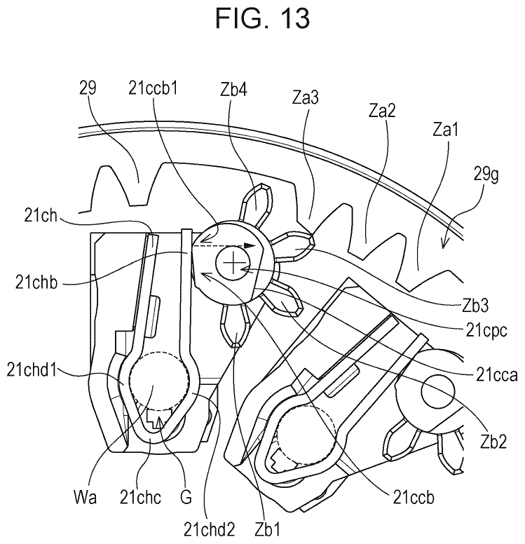

FIG. 13 is a view that shows a state where the pressing member 21cp is rotated as a result of rotation of the operating portion 400 in the lock direction.

As shown in FIG. 13, when the operating portion 400 is further rotated from the state shown in FIG. 12 in the lock direction, the internal gear 29 is further rotated in the clockwise direction.

The internal gear 29 shifts from the state of FIG. 12 to the state of FIG. 13, with the result that the internal gear 29 rotates the gear portion 21cg in the clockwise direction. When the gear portion 21cg rotates, the holding surface 21cca separates from the pressed portion 21chb, and the pressing surface 21ccb approaches the pressed portion 21chb. Then, the first part 21chd1 and the second part 21chd2 begin to clamp the held portion Wa.

Then, while the pressed portion 21chb is pressed by a corner 21ccb1 disposed at the end portion of the pressing surface 21ccb, a tooth Za3 of the internal gear 29 moves to a position separated from a tooth Zb3 of the gear portion 21cg. At this time, the held portion Wa is clamped by the first part 21chd1 and the second part 21chd2.

When the tooth Za3 of the internal gear 29 separates from the tooth Zb3 of the gear portion 21cg, transmission of driving force from the internal gear 29 to the gear portion 21cg stops. At this time, the corner 21ccb1 of the cam 21cc receives a reaction force from the leaf spring 21ch.

In the radial direction of rotation of the pressing member 21cp, the reaction force of the leaf spring 21ch, applied to the corner 21ccb1, is applied at a position spaced apart from the rotation center 21cpc of the pressing member 21cp, and the pressing member 21cp rotates in the clockwise direction. At this time, the pressing member 21cp rotates in the same direction as the direction to be rotated by the internal gear 29 that rotates in the clockwise direction.

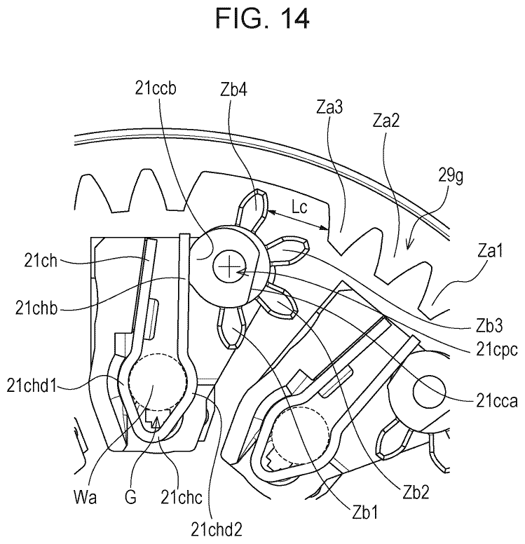

FIG. 14 is a view that shows a state of the internal gear 29 and the coupling portion 21c in a state where the operating portion 400 is at the fixed position.

As shown in FIG. 14, the pressing member 21cp further rotates from the state shown in FIG. 13 upon receiving the reaction force of the leaf spring 21ch.

As shown in FIG. 14, the pressing member 21cp stops in a state where the pressing surface 21ccb of the cam 21cc and the pressed portion 21chb of the leaf spring 21ch are in area contact with each other. In other words, the pressing surface 21ccb and the surface of the pressed portion 21chb are in a state of being arranged in the same plane.

At this time, the coupling portion 21c is in a locked state. When the coupling portion 21c is in the locked state, the cam 21cc of the pressing member 21cp is placed at the pressing position, and the pressing surface 21ccb presses the pressed portion 21chb.

When the coupling portion 21c is in the locked state, the held portion Wa is clamped by the first part 21chd1 and the second part 21chd2. In other words, the leaf spring 21ch is pressed by the cam 21cc, and the held portion Wa is fastened by the leaf spring 21ch. As a result, the held portion Wa is fixed by the leaf spring 21ch.

In this way, when the catheter unit 100 is attached to the base unit 200 and then the operating portion 400 moves to the fixed position, the state becomes the attachment completed state.

In the present embodiment, in the leaf spring 21ch, the first part 21chd1 and the second part 21chd2 press the held portion Wa at positions spaced apart from each other. In addition, a bent portion 21chc is disposed between the first part 21chd1 and the second part 21chd2 to connect the first part 21chd1 and the second part 21chd2. The bent portion 21chc is disposed with a gap G from the held portion Wa. Thus, the held portion Wa is stably fixed by the first part 21chd1 and the second part 21chd2.

The material of the leaf spring 21ch may be a resin or a metal and is preferably a metal.

When the coupling portion 21c is in the locked state, the held portion Wa is restricted from being pulled out from between the first part 21chd1 and the second part 21chd2.

The tooth Za3 of the internal gear 29 and a tooth Zb4 of the gear portion 21cg are stopped at positions where there is a clearance Lc therebetween.

When fixing of the drive wire W with the coupling portion 21c is unlocked, the operating portion 400 at the fixed position is rotated in the unlock direction. At this time, the internal gear 29 rotates from the state shown in FIG. 14 in the counterclockwise direction. When the internal gear 29 rotates in the counterclockwise direction, the tooth Za3 of the internal gear 29 contacts with the tooth Zb4 of the gear portion 21cg, and the pressing member 21cp is rotated in the counterclockwise direction.

By further rotating the internal gear 29 in the counterclockwise direction, fixing of the drive wire W with the coupling portion 21c is unlocked. The operations of the internal gear 29 and the pressing member 21cp at this time are operations reverse to the above-described operations. In other words, fixing of the drive wire W with the coupling portion 21c is unlocked by the operation reverse to the operation at the time of fixing the above-described drive wire W with the coupling portion 21c.

The above-described operations are performed in each of the first to ninth coupling portions (21c11 to 21c33). In other words, in the course in which the operating portion 400 moves from the detachment position to the fixed position, the first to ninth coupling portions (21c11 to 21c33) shift from the unlocked state to the locked state by movement (rotation) of the operating portion 400. In the course in which the operating portion 400 moves from the fixed position to the detachment position, the first to ninth coupling portions (21c11 to 21c33) shift from the locked state to the unlocked state by movement (rotation) of the operating portion 400. In this way, with an operation to rotate the one operating portion 400, it is possible to switch the first to ninth coupling portions (21c11 to 21c33) between an unlocked state and a locked state.

In other words, it is not necessary that each of the plurality of coupling portions 21c includes an operating portion for switching between the unlocked state and the locked state and a user operates the plurality of coupling portions. Therefore, a user is able to easily attach and detach the catheter unit 100 to the base unit 200. In addition, the medical device 1 is simplified.

A state where each of the first to ninth drive wires (W11 to W33) is fixed by a corresponding one of the first to ninth coupling portions (21c11 to 21c33) is referred to as a first state. A state where fixing of each of the first to ninth drive wires (W11 to W33) with a corresponding one of the first to ninth coupling portions (21c11 to 21c33) is unlocked is referred to as a second state.

Interlocking with the movement of the operating portion 400, the state is switched between the first state and the second state. In other words, interlocking with the movement of the operating portion 400 between the detachment position and the fixed position, the state is switched between the first state and the second state.

The internal gear 29 is configured to interlock with the operating portion 400. In the present embodiment, the joint 28 functions as a transmission member for interlocking the operating portion 400 with the internal gear 29. The internal gear 29 and the joint 28 function as an interlocking portion that interlocks with the operating portion 400 such that the state switches between the first state and the second state interlocking with the movement of the operating portion 400.

Specifically, the internal gear 29 and the joint 28 move part (pressed portion 21chb) of the leaf spring 21ch toward the held portion Wa interlocking with the movement of the operating portion 400 in a state where the catheter unit 100 is attached to the base unit 200. When the held portion 21chb moves, the coupling portion 21c is switched between the locked state and the unlocked state.

Alternatively, the internal gear 29 may be configured to be directly moved by the operating portion 400. In this case, the internal gear 29 has the function of the interlocking portion.

### Movement of Operating Portion and Fixation Detection

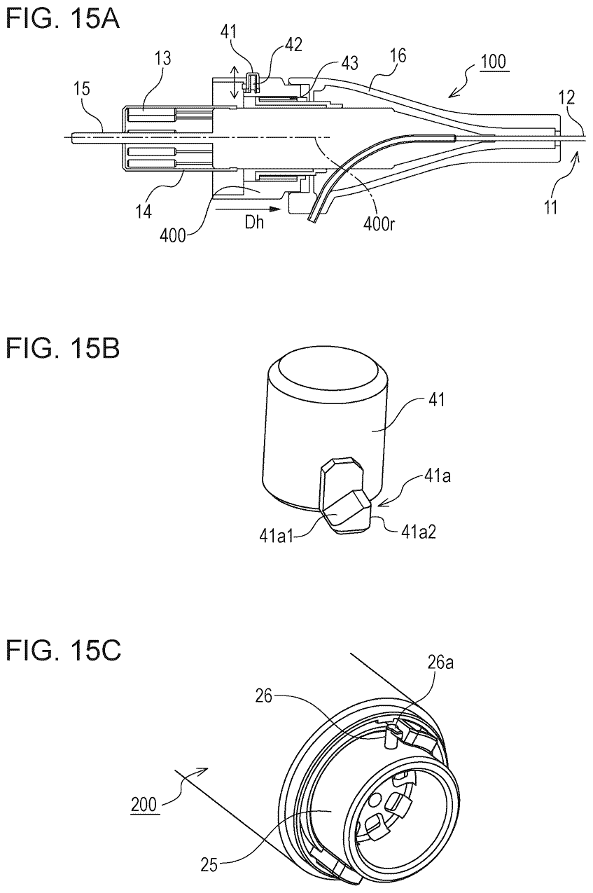

Movement of the operating portion 400 and fixation detection will be described with reference to FIGS. 15A, 15B, 15C, 16A, 16B, 16C, 17A, 17B, 17C, 18A, 18B, and 18C.

In the present embodiment, the operating portion 400 is configured to be movable among the detachment position, the unlock position, and the fixed position in a state where the catheter unit 100 is attached to the base unit 200. The unlock position is located between the detachment position and the fixed position.

In the present embodiment, the operating portion 400 interlocks with the movement of the operating portion 400 between the unlock position and the fixed position, and the state is switched between the first state and the second state.

In the present embodiment, the operating portion 400 is movable between the detachment position and the fixed position by moving in a direction different from the attaching and detaching direction DE. The operating portion 400 moves in a direction that intersects with (preferably, a direction orthogonal to) the attaching and detaching direction DE to move between the detachment position and the fixed position. In the present embodiment, the operating portion 400 rotates around the rotation axis 400r extending in the attaching and detaching direction DE to move between the detachment position and the fixed position. Therefore, operability at the time when a user operates the operating portion 400 is good.

FIGS. 15A, 15B, and 15C are views that illustrate the catheter unit 100 and the base unit 200. FIG. 15A is a sectional view of the catheter unit 100. FIG. 15B is a perspective view of a button 41. FIG. 15C is a perspective view of the base unit 200.

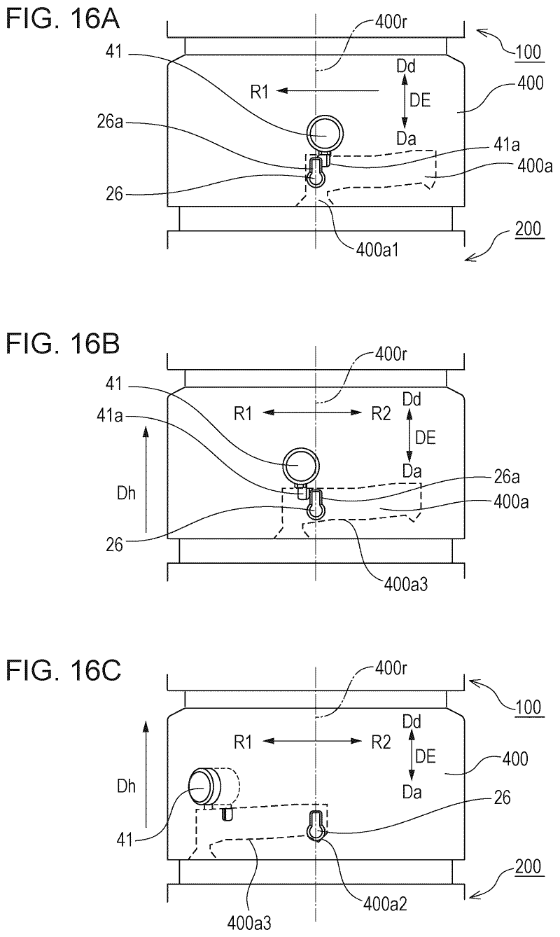

FIGS. 16A, 16B, and 16C are views that illustrate the operation of the operating portion 400 when viewed from the connection shaft 26c side. FIG. 16A is a view that shows a state where the operating portion 400 is at the detachment position. FIG. 16B is a view that shows a state where the operating portion 400 is at the unlock position. FIG. 16C is a view that shows a state where the operating portion 400 is at the fixed position.

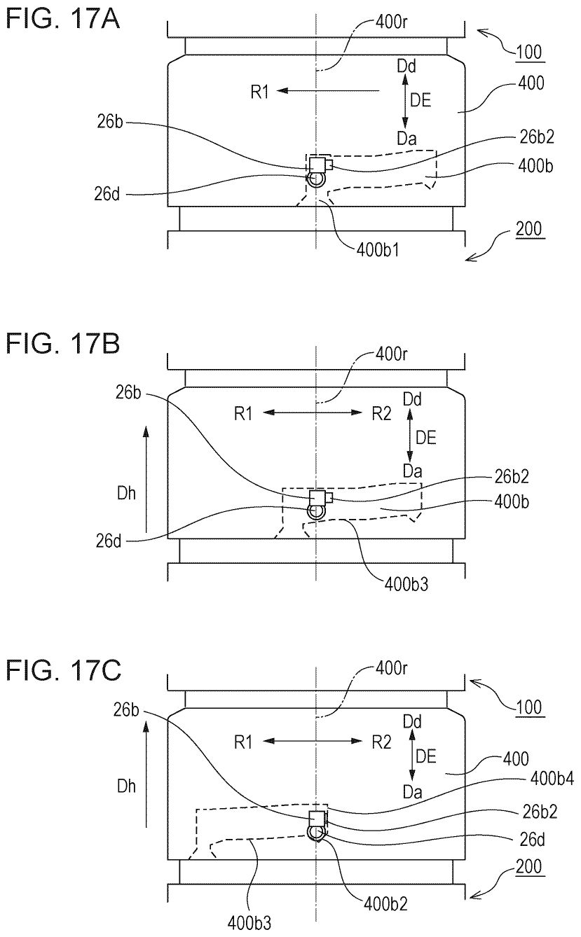

FIGS. 17A, 17B, and 17C are views that illustrate the operation of the operating portion 400 when viewed from the connection shafts 26d side. FIG. 17A is a view that shows a state where the operating portion 400 is at the detachment position. FIG. 17B is a view that shows a state where the operating portion 400 is at the unlock position. FIG. 17C is a view that shows a state where the operating portion 400 is at the fixed position.

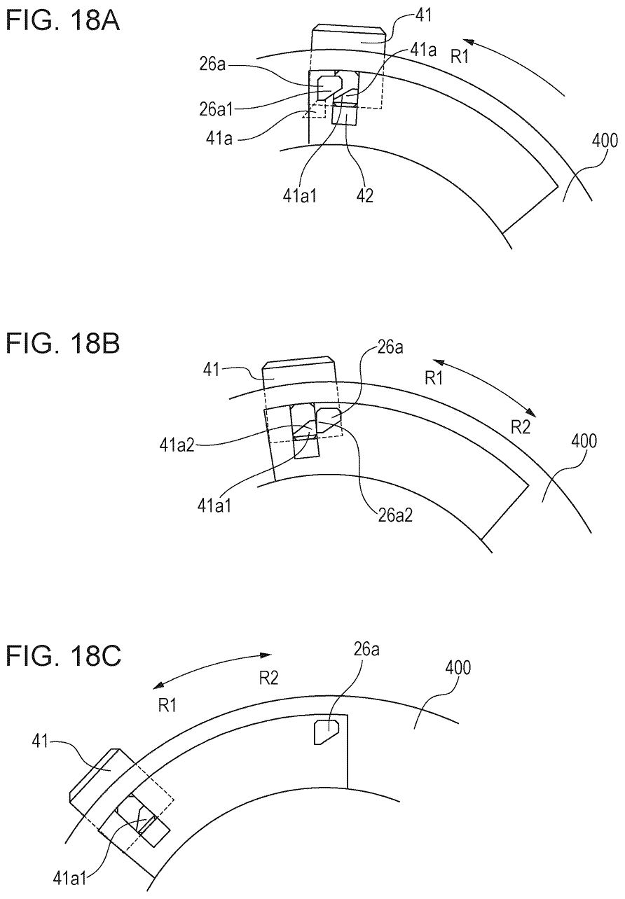

FIGS. 18A, 18B, and 18C are sectional views that illustrate the operation of the operating portion 400. FIG. 18A is a sectional view that shows a state where the operating portion 400 is at the detachment position. FIG. 18B is a sectional view that shows a state where the operating portion 400 is at the unlock position. FIG. 18C is a sectional view that shows a state where the operating portion 400 is at the fixed position.

When the operating portion 400 is at the fixed position, the coupling portion 21c is in the locked state, and the held portion Wa of the drive wire W is fixed to the corresponding coupling portion 21c (see FIG. 14).

When the operating portion 400 is at the unlock position, the coupling portion 21c is in the unlocked state, and the locking of the held portion Wa of the drive wire W with the coupling portion 21c is unlocked (see FIG. 11). In this state, connection of the drive wire W with the wire drive portion 300 is interrupted. Therefore, at the time when the catheter 11 receives an external force, the bend portion 12 can be freely bent without receiving resistance from the wire drive portion 300.

When the operating portion 400 is at the detachment position, the catheter unit 100 is allowed to be detached from the base unit 200. In a state where the operating portion 400 is at the detachment position, the catheter unit 100 is allowed to be attached to the base unit 200. When the operating portion 400 is at the detachment position, the coupling portion 21c is in the unlocked state, and the locking of the held portion Wa of the drive wire W with the coupling portion 21c is unlocked (see FIG. 10).

As shown in FIG. 15A, the catheter unit 100 includes an operating portion urging spring 43 that urges the operating portion 400, the button 41 serving as a moving member, and a button spring 42 that urges the button 41.

In the present embodiment, the operating portion urging spring 43 is a compression spring. The operating portion 400 is urged in a direction Dh to approach the proximal end cover 16 by the operating portion urging spring 43.

In the present embodiment, the button 41 and the button spring 42 are included in the operating portion 400. When the operating portion 400 moves among the detachment position, the unlock position, and the fixed position, the button 41 and the button spring 42 move together with the operating portion 400.

The button 41 is configured to be movable with respect to the operating portion 400 in a direction that intersects with the direction of the rotation axis 400r of the operating portion 400. The button 41 is urged by the button spring 42 toward outside the catheter unit 100 (a direction to move away from the rotation axis 400r).

As will be described later, movement of the operating portion 400 from the unlock position to the detachment position is restricted by the button 41. When the button 41 is moved with respect to the operating portion 400, the operating portion 400 is allowed to move from the unlock position to the detachment position.

The button 41 has a button protrusion (regulated portion) 41a. The button protrusion 41a has a button slope 41a1 and a restricted surface 41a2.

The base unit 200 includes the base frame 25. The base frame 25 is provided with the two connection shafts 26c, 26d. The connection shaft 26c has a lock protrusion (regulating portion) 26a. The connection shaft 26d includes the fixation detection sensor 26b. In the present embodiment, the fixation detection sensor 26b is a push down switch and is fixation detection means that detects whether the operating portion 400 is at the fixed position.

An example in which the two connection shafts 26c, 26d are provided has been described. Alternatively, a plurality of connection shafts may be provided and each may have a lock protrusion 26a, or one or some connection shafts may have a lock protrusion 26a. One fixation detection sensor 26b just needs to be provided, and one connection shaft may include both the lock protrusion 26a and the fixation detection sensor 26b.

On the other hand, as shown in FIGS. 9, 16A, 16B, 16C, 17A, 17B, and 17C, a connection groove 400a to be engaged with the connection shaft 26c and a detection groove 400b to be engaged with the connection shaft 26d are provided on the inner side of the operating portion 400. The connection groove 400a and the detection groove 400b extend in a direction different from the attaching and detaching direction DE. In the present embodiment, the connection groove 400a and the detection groove 400b extend in the rotation direction of the operating portion 400. The connection groove 400a and the detection groove 400b may also be regarded as extending in a direction that intersects with (direction orthogonal to) the attaching and detaching direction DE.

When a plurality of connection shafts is provided, the connection groove 400a and the detection groove 400b are selectively respectively provided for the plurality of connection shafts.

As shown in FIG. 16A, when the catheter unit 100 is attached to the base unit 200, the connection shaft 26c engages with the connection groove 400a via the entrance 400a1 of the connection groove 400a. Similarly, as shown in FIG. 17A, the connection shaft 26d engages with the detection groove 400b via the entrance 400b1 of the detection groove 400b.

At this time, the operating portion 400 is at the detachment position, and the coupling portion 21c is in the unlocked state (see FIG. 10). Therefore, this is a state where fixing of each of the first to ninth drive wires (W11 to W33) with a corresponding one of the first to ninth coupling portions (21c11 to 21c33) is unlocked. At this time, as shown in FIG. 17A, a button 26b2 of the fixation detection sensor 26b is in an open state (OFF). Therefore, the controller 3 can recognize that the coupling portion 21c is in the unlocked state. As shown in FIG. 18A, the button protrusion 41a is opposite to the lock protrusion 26a.

When the operating portion 400 is rotated in a lock direction R1 in a state where the operating portion 400 is at the detachment position, the slope 41a1 of the button protrusion 41a contacts with a slope 26a1 of the lock protrusion 26a. The button 41 moves toward inside the operating portion 400 (a direction to approach the rotation axis 400r) against the urging force of the button spring 42. Then, the button protrusion 41a climbs over the lock protrusion 26a, and the operating portion 400 moves to the unlock position (see FIG. 18B).

As shown in FIG. 17B, the connection shaft 26d moves a distance equivalent to a distance the operating portion 400 has moved in the lock direction R1 in the detection groove 400b.

At this time, the coupling portion 21c is in the unlocked state (see FIG. 11). Therefore, this is a state where fixing of each of the first to ninth drive wires (W11 to W33) with a corresponding one of the first to ninth coupling portions (21c11 to 21c33) is unlocked. At this time, the button 26b2 of the fixation detection sensor 26b is in the open state (OFF). Therefore, the controller 3 can recognize that the coupling portion 21c is in the unlocked state.

In the present embodiment, even when the button 41 is not operated, the operating portion 400 is allowed to be moved from the detachment position to the unlock position. In other words, when the operating portion 400 is moved from the detachment position to the unlock position, a user does not need to operate the button 41.

When the operating portion 400 is rotated in the lock direction R1 in a state where the operating portion 400 is at the unlock position, the operating portion 400 moves to the fixed position. When the operating portion 400 is at the fixed position, a positioning portion 400a2 of the connection groove 400a is at a location corresponding to the connection shaft 26c. Similarly, when the operating portion 400 is at the fixed position, a positioning portion 400b2 of the detection groove 400b is at a location corresponding to the connection shaft 26d. The operating portion 400 is urged in a direction Dh to approach the proximal end cover 16 by the operating portion urging spring 43. As a result, the positioning portion 400a2 engages with the connection shaft 26c, and the positioning portion 400b2 engages with the connection shaft 26d.

In the course in which the operating portion 400 moves from the unlock position to the fixed position, the held portion Wa of the drive wire W is fixed to the coupling portion 21c as described above.

In a state where the operating portion 400 is at the fixed position, the coupling portion 21c is in the locked state (see FIG. 14). Therefore, each of the first to ninth drive wires (W11 to W33) is fixed to a corresponding one of the first to ninth coupling portions (21c11 to 21c33). In this state, driving force from the wire drive portion 300 can be transmitted to the bend drive portion 13. In other words, driving force from each of the first to ninth driving sources (M11 to M33) can be transmitted to a corresponding one of the first to ninth drive wires (W11 to W33) via a corresponding one of the first to ninth coupling portions (21c11 to 21c33).

At this time, as shown in FIG. 17C, the button 26b2 of the fixation detection sensor 26b is in the pressed down state (ON) by a wall 400b4 forming the detection groove 400b. Therefore, the controller 3 can recognize that the coupling portion 21c is in the locked state.

In a state where the operating portion 400 is in the unlock position, a wall 400a3 forming the connection groove 400a and a wall 400b3 forming the detection groove 400b each are located on the upstream side of the connection shaft 26 in the detachment direction Dd of the catheter unit 100. When the operating portion 400 is at the fixed position, the positioning portion 400a2 is located on the upstream side of the connection shaft 26c and the positioning portion 400b2 is located on the upstream side of the connection shaft 26d in the detachment direction Dd. As a result, when the operating portion 400 is at the unlock position or at the fixed position, detaching the catheter unit 100 from the base unit 200 is restricted. On the other hand, when the operating portion 400 is at the detachment position, the entrance 400a1 of the connection groove 400a is located on the upstream side of the connection shaft 26c and the entrance 400b1 of the detection groove 400b is located on the upstream side of the connection shaft 26d in the detachment direction Dd. As a result, detaching the catheter unit 100 from the base unit 200 is allowed.

When the operating portion 400 is rotated in an unlock direction R2 in a state where the operating portion 400 is at the fixed position, the operating portion 400 is placed at the unlock position. In the course in which the operating portion 400 moves from the fixed position to the unlock position, the held portion Wa of the drive wire W is unlocked from the coupling portion 21c as described above.

The button 26b2 of the fixation detection sensor 26b is in the open state (OFF) to separate from the wall 400b4 forming the detection groove 400b. Therefore, the controller 3 can recognize that the coupling portion 21c is in the unlocked state.

In a state where the operating portion 400 is at the unlock position, the restricted surface 41a2 of the button protrusion 41a contacts with the restriction surface 26a2 of the lock protrusion 26a (see FIG. 18B). In this state, rotating the operating portion 400 in the unlock direction R2 is restricted. Detaching the catheter unit 100 from the base unit 200 is restricted.

When a user pushes the button 41 toward inside the operating portion 400 in a state where the operating portion 400 is at the unlock position, the restricted surface 41a2 separates from the restriction surface 26a2, and the button protrusion 41a climbs over the lock protrusion 26a. As a result, the operating portion 400 is allowed to rotate in the unlock direction R2, and the operating portion 400 can be moved from the unlock position to the detachment position.

In a state where the operating portion 400 is at the detachment position, the coupling portion 21c is in the unlocked state. Therefore, when the catheter unit 100 is detached from the base unit 200 or attached to the base unit 200, load (for example, resistance received by the coupling portion 21c) applied to the drive wire W is reduced. Therefore, a user is able to easily attach and detach the catheter unit 100.

In a state where the operating portion 400 is at the unlock position, detaching the catheter unit 100 from the base unit 200 is restricted, and the coupling portion 21c is in the unlocked state. As described above, when the coupling portion 21c is in the unlocked state, connection of the drive wire W with the wire drive portion 300 is interrupted, and the bend portion 12 can be freely bent without receiving resistance from the wire drive portion 300.

A user is able to stop actuation of the catheter 11 with the wire drive portion 300 by placing the operating portion 400 at the unlock position in a state where the catheter 11 is inserted in a target. Since detaching the catheter unit 100 from the base unit 200 is restricted, a user is able to pull out the catheter 11 from inside the target while holding the base unit 200.

In the configuration of the present embodiment, when the button 41 is not operated, movement of the operating portion 400 from the unlock position to the detachment position is restricted. Therefore, when a user moves the operating portion 400 from the fixed position to the unlock position, erroneous movement of the operating portion 400 to the detachment position is reduced.

In the present embodiment, the number of the lock protrusions 26a and the number of the buttons 41 are one of each. However, the medical device 1 may have a plurality of the lock protrusions 26a and a plurality of the buttons 41.

In the present embodiment, an example in which the fixation detection sensor 26b is a push down switch has been described; however, the configuration is not limited thereto. For example, a configuration in which transmission and shielding are switched by movement of the operating portion 400 by using a transmissive optical sensor, a sensor flag, and a light shielding wall may be applied. A configuration in which a reflective optical sensor and a reflector are used and the relationship in opposite arrangement between the reflective optical sensor and the reflector varies as a result of movement of the operating portion 400 may be applied. Alternatively, a pressure sensor that detects a pressure that occurs as a result of movement of the operating portion 400 may be applied. Alternatively, a configuration in which a spring contact and a contact plate are provided and the spring contact and the contact plate contact with each other to obtain electrical continuity as a result of movement of the operating portion 400 and transmit a signal may be applied. Alternatively, a configuration in which a rotational phase of the operating portion 400 is detected with an encoder may be applied.

Hereinafter, a procedure at the time of using the medical system 1A and a process that the controller 3 executes will be described.

### Use Procedure of Medical System

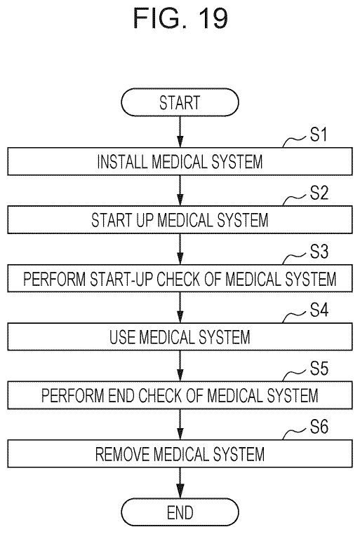

A use procedure of the medical system 1A will be described with reference to FIG. 19.

In step S1, a user installs the medical system 1A at a location where the catheter 11 can be inserted into a target. Specifically, a user installs the medical system 1A at a location where the catheter 11 can be inserted into the bronchus via the oral cavity or nasal cavity of a patient.

After installation of the medical system 1A is complete, the user starts up the medical system 1A by turning on the power of the medical system 1A in step S2.

After the startup of the medical system 1A, the user performs start-up check for using the medical system 1A in step S3. At this time, the controller 3 is configured to execute necessary processes, including checking attachment of the catheter unit 100 with the base unit 200 (described later), for using the medical system 1A (step S7). Specifically, the controller 3 checks the states and operations of sensors, units, and software for using the medical system 1A.

After the start-up check, the user in step S4 operates the medical device 1 and performs work, such as observing the inside of the target, collecting various samples from the inside of the target, and treating the inside of the target, by inserting the catheter 11 into the inside of the target.

After use of the medical system 1A, the user in step S5 performs end check for terminating the medical system 1A. At this time, the controller 3 is configured to execute necessary processes, including checking detachment of the catheter unit 100 from the base unit 200 (described later), for terminating the medical system 1A (step S8). Specifically, the controller 3 checks the states and operations of sensors, units, and software for terminating the medical system 1A.

After the end check, the user in step S6 removes the medical system 1A as needed.

Through instructions of the user, the controller 3 can skip the start-up check (step S3) or the end check (step S5) according to the previous end state. However, even when the controller 3 skips the start-up check or the end check through determination of the user, the controller 3 monitors the states of sensors and the like disposed at locations and, when there is a defect, notifies that information to the user.

Checking of Attachment of Catheter Unit with Base Unit

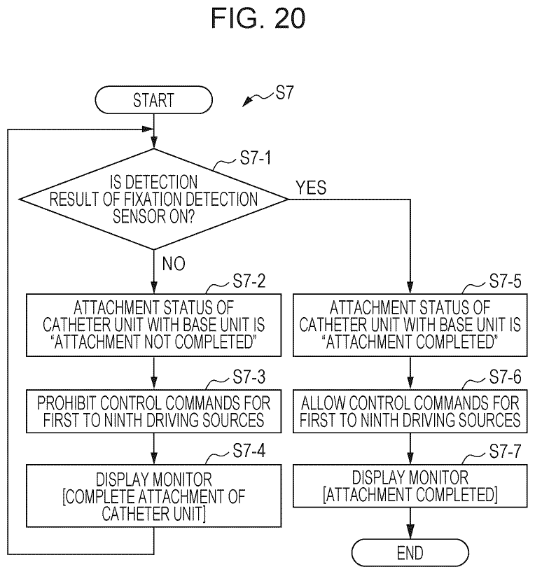

Checking of attachment of the catheter unit 100 with the base unit 200 (step S7: hereinafter, referred to as checking of attachment) in the first embodiment will be described with reference to FIG. 20.

Checking of attachment can be performed at selected timing. In the present embodiment, checking of attachment is performed during execution of the start-up check (step S3) or when it is necessary to perform re-setup due to some system trouble. For example, the controller 3 performs checking of attachment in accordance with instructions to start attachment of the catheter unit 100 with the base unit 200. The controller 3 just needs to be able to be instructed to start checking of attachment by the user pushing an attachment start button (not shown) provided in the medical system 1A. Instructions to start attachment may be any manner with which the user can transfer instructions to the controller 3 and may be, for example, a manner to select in an operation screen shown on the monitor 4. During execution of the start-up check (step S3), the controller 3 may automatically perform checking of attachment.

In step S7-1, the controller 3 checks the detection result of the fixation detection sensor 26b. When the detection result of the fixation detection sensor 26b is the open state (OFF), the process proceeds to step S7-2; whereas, when the detection result is the pressed down state (ON), the process proceeds to step S7-5.

In step S7-2, the controller 3 determines that attachment is not completed, that is, attachment of the catheter unit 100 with the base unit 200 is not completed (attachment not completed).

In step S7-3, the controller 3 prohibits control input to the first to ninth driving sources (M11 to M33). At this time, for example, control input to components and units, which can be factors of failure, such as a motor (not shown) that moves the movable stage 2a, may also be prohibited. In this way, during a period from a time point when the medical system 1A is started up to completion of attachment, the controller 3 prohibits control of the first to ninth driving sources (M11 to M33). By prohibiting control over the first to ninth driving sources (M11 to M33), a failure or the like of the device due to unintended actuation of the driving source in work for attachment of the catheter unit 100 with the base unit 200 or detachment work (described later).

In step S7-4, the controller 3 notifies that attachment is not completed, with, for example, the monitor 4, and the process returns to step S7-1. At this time, a message prompting to attach the catheter unit 100 to the base unit 200 or a message guiding to completion of attachment (such a message guiding to rotate the operating portion 400 in the lock direction R1 to move to the fixed position) may be notified together. A method of notifying the user may be visual information, such as LED display, or may be notification through sound or vibration. The same also applies to notifications described below. The user checks the notification from the controller 3 and attaches the catheter unit 100 to the base unit 200.

The controller 3 repeats a series of processes of step S7-1 to step S7-4 until the detection result of the fixation detection sensor 26b becomes the pressed down state (ON).

In step S7-5, the controller 3 determines that attachment of the catheter unit 100 with the base unit 200 is completed (attachment completed).

In step S7-6, the controller 3 allows control input to the first to ninth driving sources (M11 to M33).

In step S7-7, the controller 3 notifies that attachment is completed, with, for example, the monitor 4, and ends the flowchart.

At the time of starting checking of attachment in step S7, attachment of the catheter unit 100 with the base unit 200 may be completed. This is, for example, a case where work for attaching the catheter unit 100 to the base unit 200 is performed before the start-up check (step S3) is started. Alternatively, this is, for example, a case where, at the previous termination of the medical system 1A, detachment of the catheter unit 100 from the base unit 200 has not been performed. In this case, the detection result of the fixation detection sensor 26b becomes the pressed down state (ON) in the initial determination process (step S7-1), and the process immediately proceeds to step S7-5.

Checking of Detachment of Catheter Unit from Base Unit

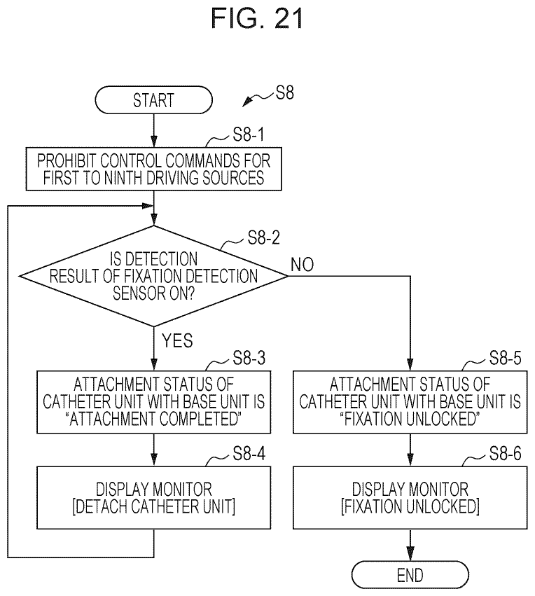

Checking of detachment of the catheter unit 100 from the base unit 200 (step S8: hereinafter, referred to as checking of detachment) in the first embodiment will be described with reference to FIG. 21.

Checking of detachment can be performed at selected timing. In the present embodiment, checking of detachment is performed during execution of the end check (step S5) or when it is necessary to withdraw the device due to some system trouble. For example, the controller 3 performs checking of detachment in accordance with instructions to start detachment of the catheter unit 100 from the base unit 200. The controller 3 just needs to be able to be instructed to start checking of detachment by the user pushing a detachment start button (not shown) provided in the medical system 1A. Instructions to start detachment may be any manner with which the user can transfer instructions to the controller 3 and may be, for example, a manner to select in an operation screen shown on the monitor 4. During execution of the end check (step S5) or in an emergency stop, the controller 3 may automatically perform checking of detachment.

In step S8-1, the controller 3 prohibits control input to the first to ninth driving sources (M11 to M33). At this time, for example, control input to components and units, which can be factors of failure, such as a motor (not shown) that moves the movable stage 2a, may also be prohibited.

In step S8-2, the controller 3 checks the detection result of the fixation detection sensor 26b. When the detection result of the fixation detection sensor 26b is the pressed down state (ON), the process proceeds to step S8-3; whereas, when the detection result is the open state (OFF), the process proceeds to step S8-5.

In step S8-3, the controller 3 determines that attachment of the catheter unit 100 with the base unit 200 is completed (attachment completed), that is, fixation is not unlocked.

In step S8-4, the controller 3 notifies that attachment is completed, that is, fixation is not unlocked, with, for example, the monitor 4, and the process returns to step S8-2. At this time, a message prompting to detach the catheter unit 100 from the base unit 200 or a message guiding for detachment (such as a message guiding to rotate the operating portion 400 in the unlock direction R2 from the fixed position to move to the detachment position) may be notified together.

The user checks the notification from the controller 3 and detaches the catheter unit 100 from the base unit 200.

The controller 3 repeats a series of processes of step S8-2 to step S8-4 until the detection result of the fixation detection sensor 26b becomes the open state (OFF).

In step S8-5, the controller 3 determines that attachment of the catheter unit 100 with the base unit 200 is not completed (attachment not completed), that is, fixation is unlocked.

In step S8-6, the controller 3 notifies that attachment is not completed, that is, fixation is unlocked, with, for example, the monitor 4, and ends the flowchart. At this time, a message prompting to detach the catheter unit 100 from the base unit 200 may be notified together. The user checks the notification from the controller 3 and completes detachment of the catheter unit 100 from the base unit 200.

At the time of starting checking of detachment in step S8, detachment of the catheter unit 100 from the base unit 200 may be complete. This is, for example, a case where work for detaching the catheter unit 100 from the base unit 200 is performed before the end check (step S5) is started. In this case, the detection result of the fixation detection sensor 26b becomes the open state (OFF) in the initial determination process (step S8-2), and the process immediately proceeds to step S8-5.

Monitoring of Attachment of Catheter Unit with Base Unit

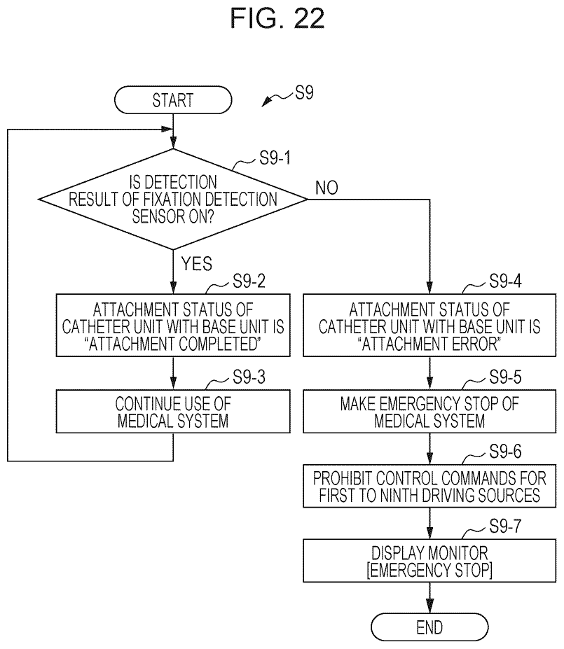

Monitoring of attachment of the catheter unit 100 with the base unit 200 (step S9: hereinafter, referred to as monitoring of attachment) in the first embodiment will be described with reference to FIG. 22.

The controller 3 performs constant attachment monitoring during use of the medical system 1A (step S4) from the end of the start-up check (step S3) to the start of the end check (step S5). Every time a certain period of time elapses or on condition that an operation is performed by the user, periodical check may be performed.

In step S9-1, the controller 3 checks the detection result of the fixation detection sensor 26b. When the detection result of the fixation detection sensor 26b is the pressed down state (ON), the process proceeds to step S9-2; whereas, when the detection result is the open state (OFF), the process proceeds to step S9-4.

In step S9-2, the controller 3 determines that attachment of the catheter unit 100 with the base unit 200 is completed (attachment completed), that is, the attachment completion state is maintained.

In step S9-3, the controller 3 allows continuous use of the medical system 1A.

In step S9-4, the controller 3 determines that attachment of the catheter unit 100 with the base unit 200 is not completed (attachment not completed), that is, the attachment completion state is not maintained, and the state is an error state (attachment error).

In step S9-5, the controller 3 makes an emergency stop of the medical system 1A.

In step S9-6, the controller 3 prohibits control input to the first to ninth driving sources (M11 to M33). At this time, for example, control input to components and units, which can be factors of failure, such as a motor (not shown) that moves the movable stage 2a, may also be prohibited.

In step S9-7, the controller 3 notifies that the medical system 1A is brought into an emergency stop due to attachment error with, for example, the monitor 4, and ends the flowchart. The user checks the notification from the controller 3, stops the operation of the medical system 1A, and withdraws the medical system 1A. In the present embodiment, a withdrawal operation by the user means to rotate the operating portion 400 in the unlock direction R2 into a second state where fixing of each of the first to ninth drive wires (W11 to W33) by a corresponding one of the first to ninth coupling portions (21c11 to 21c33) is unlocked and then move the catheter unit 100 to a location apart a certain distance from a work target. When it may be determined that safety of the user and the work target is ensured at the time of re-actuating the medical system 1A, the configuration is not limited to being apart a certain distance from the work target. The user, after withdrawal operation, is able to resume work by setting up the medical system 1A again. The user may check the notification from the controller 3 and terminate the medical system 1A without setting up again.

As described above, in the medical device 1 in which the catheter unit 100 is detachably attachable to the base unit 200, by notifying completion of attachment of the catheter unit 100 with the base unit 200 and attachment not completed, the attaching/detaching work can be assisted, so workability is improved.

### Second Embodiment

Next, a second embodiment will be described. The basic configuration and processing operations of the medical system 1A and the medical device 1 are similar to those of the first embodiment. Hereinafter, like reference signs denote components common to those of the first embodiment, the description thereof is omitted, and differences from the first embodiment will be mainly described.

### Attachment of Catheter Unit and Attachment Detection

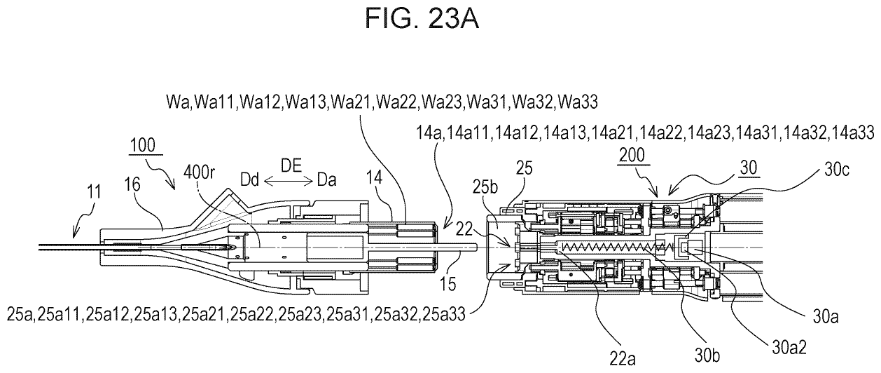

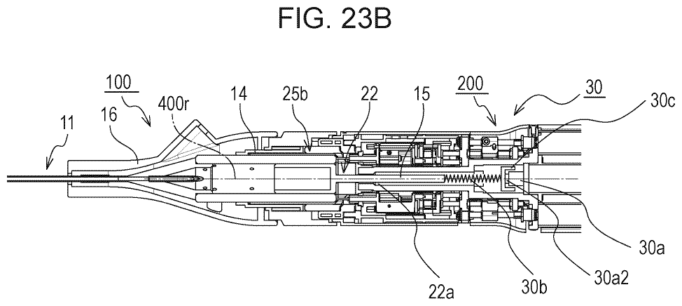

An operation to attach the catheter unit 100 to the base unit 200 will be described with reference to FIGS. 23A and 23B.

FIGS. 23A and 23B are views that illustrate attachment of the catheter unit 100. FIG. 23A is a view before the catheter unit 100 is attached to the base unit 200. FIG. 23B is a view after the catheter unit 100 is attached to the base unit 200.

An attachment detection portion 30 including an attachment detection sensor 30a, a passive spring 30b, and a passive plate 30c is provided inside the key hole 22a forming the key receiving portion 22. The attachment detection portion 30 is attachment detection means that detects whether the catheter unit 100 is attached to the base unit 200. The passive spring 30b expands and contracts with movement of the key shaft 15. The passive plate 30c receives reaction force resulting from compression of the passive spring 30b and pushes down a button 30a2 of the attachment detection sensor 30a. In this way, the attachment detection portion 30 detects attachment of the catheter unit 100 when the key shaft 15 enters the key hole 22a.

The attachment detection portion 30 is not limited to arrangement in the key receiving portion 22. The attachment detection portion 30 just needs to be placed at a location where, at the time when the catheter unit 100 is attached to the base unit 200, the attachment detection sensor 30a can detect attachment of the catheter unit 100 and the detection result is not affected by movement of the operating portion 400.

In the present embodiment, the attachment detection sensor 30a is a push down switch. The attachment detection sensor 30a is fixed to the base unit 200. The passive spring 30b is a compression spring. The passive spring 30b is movably disposed in the space defined by the passive plate 30c and an inner wall forming the key hole 22a, and the passive spring 30b has a free length in a state where the key shaft 15 is not engaged with the key receiving portion 22. Therefore, a pressing force pushing down the button 30a2 of the attachment detection sensor 30a is not generated, so the attachment detection sensor 30a is in the open state (OFF). At this time, the operating portion 400 is at the detachment position, so the detection result of the fixation detection sensor 26b is in the open state (OFF).

In a case where the detection result of the attachment detection sensor 30a is in the open state (OFF) and the detection result of the fixation detection sensor 26b is in the open state (OFF), the controller 3 recognizes this state as an unattached state where the catheter unit 100 is not attached to the base unit 200.

When the key shaft 15 enters into the key hole 22a so that the catheter unit 100 is attached to the base unit 200, the passive spring 30b is compressed in the space defined by the key shaft 15, the passive plate 30c, and the inner wall forming the key hole 22a. The passive plate 30c moves in the key hole 22a in the attachment direction Da upon receiving reaction force resulting from compression of the passive spring 30b and pushes down the button 30a2 of the attachment detection sensor 30a. When the attachment detection sensor 30a is in the pressed down state (ON), the controller 3 detects that the catheter unit 100 is attached.

When the user moves the operating portion 400 in a state where the catheter unit 100 is attached to the base unit 200, attachment of the catheter unit 100 with the base unit 200 is completed. A state where the catheter unit 100 is attached, the operating portion 400 is not at the fixed position (that is, the operating portion 400 is at the detachment position or the unlock position), and the coupling portion 21c is in the unlocked state is called a temporarily attached state. A state where the catheter unit 100 is attached, the operating portion 400 is at the fixed position, and the coupling portion 21c is in the locked state is called an attachment completed state.

On the other hand, in a case where the detection result of the attachment detection sensor 30a is in the open state (OFF) and the detection result of the fixation detection sensor 26b is in the pressed down state (ON), this means that the catheter unit 100 is not attached to the base unit 200 but the coupling portion 21c is in the locked state by the operating portion 400. In the present embodiment, since the catheter unit 100 includes the operating portion 400, pressing down of the fixation detection sensor 26b by the operating portion 400 is impossible in a state where the catheter unit 100 is detached from the base unit 200. In a case where the detection result of the attachment detection sensor 30a is in the open state (OFF) and the detection result of the fixation detection sensor 26b is in the pressed down state (ON), the controller 3 recognizes this state as an unexpected state, that is, an attachment error state.

In the present embodiment, an example in which each of the attachment detection sensor 30a and the fixation detection sensor 26b is a push down switch has been described; however, the configuration is not limited thereto. For example, a configuration in which attachment of the catheter unit 100 and transmission and shielding are switched by movement of the operating portion 400 by using a transmission optical sensor, a sensor flag, and a light shielding wall may be applied. A configuration in which a reflective optical sensor and a reflector are used and the relationship in opposite arrangement between the reflective optical sensor and the reflector varies as a result of attachment of the catheter unit 100 and movement of the operating portion 400 may be applied. Alternatively, a pressure sensor that detects a pressure that occurs as a result of attachment of the catheter unit 100 and movement of the operating portion 400 may be applied. Alternatively, a configuration in which a spring contact and a contact plate are provided and the spring contact and the contact plate contact with each other to obtain electrical continuity as a result of attachment of the catheter unit 100 and movement of the operating portion 400 and transmit a signal may be applied. Alternatively, a configuration in which a rotational phase of the operating portion 400 is detected with an encoder may be applied.

Checking of Attachment of Catheter Unit with Base Unit

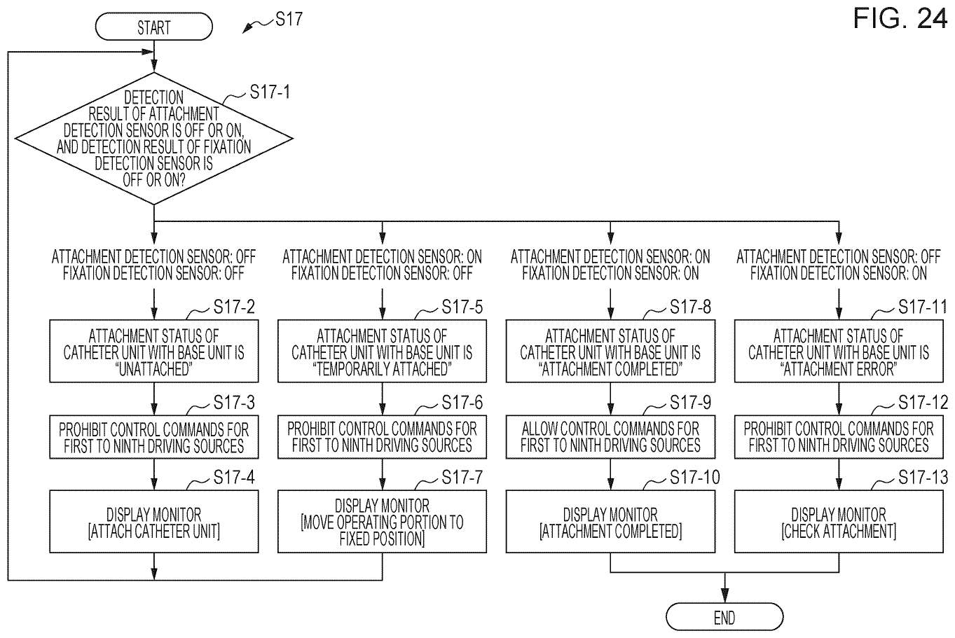

Checking of attachment of the catheter unit 100 with the base unit 200 (step S17: hereinafter, referred to as checking of attachment) in the second embodiment will be described with reference to FIG. 24.

In step S17-1, the controller 3 checks the detection result of the attachment detection sensor 30a and the detection result of the fixation detection sensor 26b.

In a case where the detection result of the attachment detection sensor 30a is in the open state (OFF) and the detection result of the fixation detection sensor 26b is in the open state (OFF) in step S17-1, the process proceeds to step S17-2.

In step S17-2, the controller 3 determines that this state is an unattached state.

In step S17-3, the controller 3 prohibits control input to the first to ninth driving sources (M11 to M33). In this way, during a period in the unattached state from a time point when the medical system 1A is started, the controller 3 prohibits control of the first to ninth driving sources (M11 to M33). By prohibiting control over the first to ninth driving sources (M11 to M33), a failure or the like of the device due to unintended actuation of the driving source in work for attachment of the catheter unit 100 with the base unit 200 or detachment work (described later). At this time, for example, control input to components and units, which can be factors of failure, such as a motor (not shown) that moves the movable stage 2a, may also be prohibited.

In step S17-4, the controller 3 notifies the unattached state with, for example, the monitor 4, and the process returns to step S17-1. At this time, a message prompting to attach the catheter unit 100 to the base unit 200 may be notified together. The user checks the notification from the controller 3 and then attaches the catheter unit 100 to the base unit 200.

In a case where the detection result of the attachment detection sensor 30a is in the pressed down state (ON) and the detection result of the fixation detection sensor 26b is in the open state (OFF) in step S17-1, the process proceeds to step S17-5.

In step S17-5, the controller 3 determines that this state is a temporarily attached state.

In step S17-6, the controller 3, as in the case of step S17-3, prohibits control input to the first to ninth driving sources (M11 to M33). At this time, for example, control input to components and units, which can be factors of failure, such as a motor (not shown) that moves the movable stage 2a, may also be prohibited.

In step S17-7, the controller 3 notifies the temporarily attached state with, for example, the monitor 4, and the process returns to step S17-1. At this time, a message guiding to completion of attachment (such a message guiding to rotate the operating portion 400 in the lock direction R1 to move to the fixed position) may be notified together. The user checks the notification from the controller 3 and completes attachment of the catheter unit 100 with the base unit 200.

In a case where the detection result of the attachment detection sensor 30a is in the pressed down state (ON) and the detection result of the fixation detection sensor 26b is in the pressed down state (ON) in step S17-1, the process proceeds to step S17-8.

In step S17-8, the controller 3 determines that this state is an attachment completed state.

In step S17-9, the controller 3 allows control input to the first to ninth driving sources (M11 to M33). At this time, for example, control input to components and units, which can be factors of failure, such as a motor (not shown) that moves the movable stage 2a, is prohibited, the controller 3 may also allow control input at the same time.

In step S17-10, the controller 3 notifies the attachment completed state with, for example, the monitor 4, and ends the flowchart.

In a case where the detection result of the attachment detection sensor 30a is in the open state (OFF) and the detection result of the fixation detection sensor 26b is in the pressed down state (ON) in step S17-1, the process proceeds to step S17-11.

In step S17-11, the controller 3 determines that this state is an attachment error state. The attachment error state is not limited to only at the time of attachment work and can be any time from startup of the medical system 1A to termination of the medical system 1A, such as at the time of work for detaching the catheter unit 100 from the base unit 200 and at the time of use of the medical system 1A.

In step S17-12, the controller 3, as in the case of step S17-3, prohibits control input to the first to ninth driving sources (M11 to M33). At this time, as in the case of handling in an emergency stop, for example, control input to components and units, which can be factors of failure, such as a motor (not shown) that moves the movable stage 2a, may also be prohibited.

In step S17-13, the controller 3 notifies the attachment error state with, for example, the monitor 4, and ends the flowchart. At this time, a message prompting to check the attachment status of the catheter unit 100 with the base unit 200 may be notified together. The user checks the notification from the controller 3 and checks the attachment status of the catheter unit 100 with the base unit 200.

Once in the attachment error state, the controller 3 may automatically execute a predetermined attachment error restoration process. Alternatively, the start of the attachment error restoration process may be waited until start instructions are received from the user. Attachment work may be performed again as attachment error restoration work. The medical system 1A may be restarted, and setup may be performed again.

Checking of Detachment of Catheter Unit from Base Unit

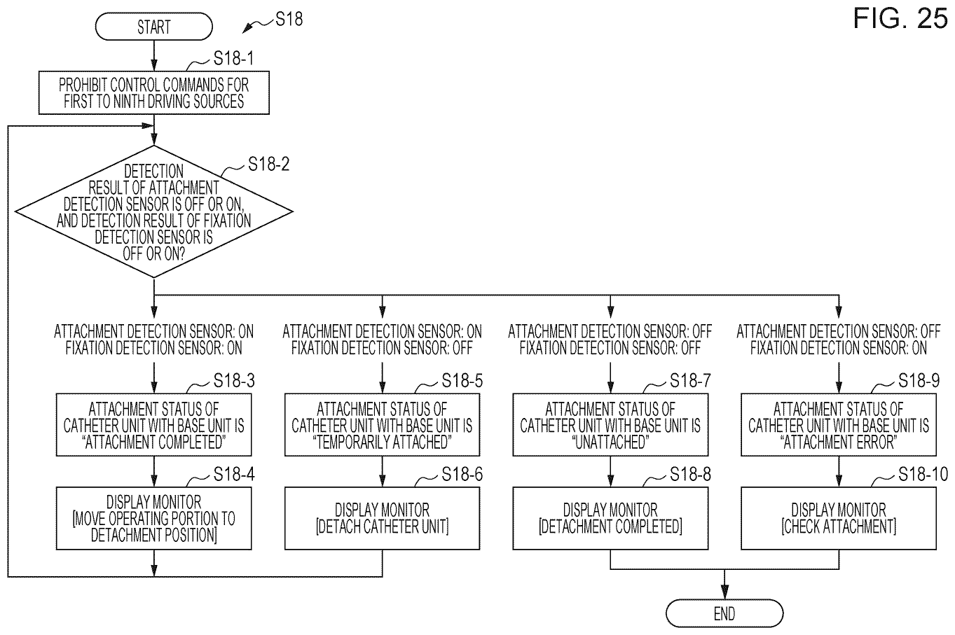

Checking of detachment of the catheter unit 100 from the base unit 200 (step S18: hereinafter, referred to as checking of detachment) in the second embodiment will be described with reference to FIG. 25.

In step S18-1, the controller 3 prohibits control input to the first to ninth driving sources (M11 to M33). At this time, for example, control input to components and units, which can be factors of failure, such as a motor (not shown) that moves the movable stage 2a, may also be prohibited.

In step S18-2, the controller 3 checks the detection result of the attachment detection sensor 30a and the detection result of the fixation detection sensor 26b.

In a case where the detection result of the attachment detection sensor 30a is in the pressed down state (ON) and the detection result of the fixation detection sensor 26b is in the pressed down state (ON) in step S18-2, the process proceeds to step S18-3.

In step S18-3, the controller 3 determines that this state is an attachment completed state.

In step S18-4, the controller 3 notifies the attachment completed state with, for example, the monitor 4, and the process returns to step S18-2. At this time, a message guiding for detachment (such as a message guiding to rotate the operating portion 400 in the unlock direction R2 from the fixed position to move to the detachment position) may be notified together. The user checks the notification from the controller 3 and moves the operating portion 400 to the detachment position.

In a case where the detection result of the attachment detection sensor 30a is in the pressed down state (ON) and the detection result of the fixation detection sensor 26b is in the open state (OFF) in step S18-2, the process proceeds to step S18-5.

In step S18-5, the controller 3 determines that this state is a temporarily attached state.

In step S18-6, the controller 3 notifies the temporarily attached state with, for example, the monitor 4, and the process returns to step S18-2. At this time, a message prompting to detach the catheter unit 100 from the base unit 200 may be notified together. Specifically, the controller 3 checks that the operating portion 400 is at the detachment position, and guides to detach the catheter unit 100 from the base unit 200. The user checks the notification from the controller 3 and detaches the catheter unit 100 from the base unit 200.

In a case where the detection result of the attachment detection sensor 30a is in the open state (OFF) and the detection result of the fixation detection sensor 26b is in the open state (OFF) in step S18-2, the process proceeds to step S18-7.

In step S18-7, the controller 3 determines that this state is an unattached state.

In step S18-8, the controller 3 notifies that this state is an unattached state, that is, detachment of the catheter unit 100 from the base unit 200 is completed, with, for example, the monitor 4, and ends the flowchart.

In a case where the detection result of the attachment detection sensor 30a is in the open state (OFF) and the detection result of the fixation detection sensor 26b is in the pressed down state (ON) in step S18-2, the process proceeds to step S18-9.

In step S18-9, the controller 3 determines that this state is an attachment error state.

In step S18-10, the controller 3 notifies the attachment error state with, for example, the monitor 4, and ends the flowchart. At this time, a message prompting to check the attachment status of the catheter unit 100 with the base unit 200 may be notified together. The user checks the notification from the controller 3 and checks the attachment status of the catheter unit 100 with the base unit 200.

Once in the attachment error state, the controller 3 may automatically execute a predetermined attachment error restoration process. Alternatively, the start of the attachment error restoration process may be waited until start instructions are received from the user.

Monitoring of Attachment of Catheter Unit with Base Unit

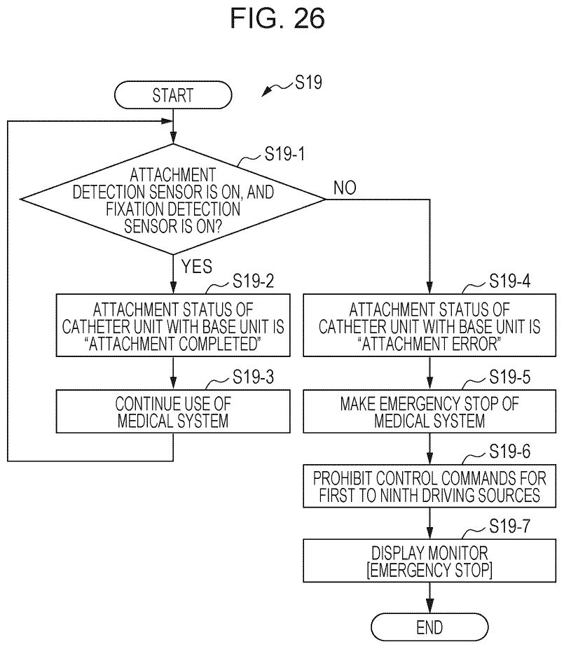

Monitoring of attachment of the catheter unit 100 with the base unit 200 (step S19: hereinafter, referred to as monitoring of attachment) in the second embodiment will be described with reference to FIG. 26.

The controller 3 performs constant attachment monitoring during use of the medical system 1A (step S4) from the end of the start-up check (step S3) to the start of the end check (step S5). Every time a certain period of time elapses or on condition that an operation is performed by the user, periodical check may be performed.

In step S19-1, the controller 3 checks the detection result of the attachment detection sensor 30a and the detection result of the fixation detection sensor 26b.

In a case where the detection result of the attachment detection sensor 30a is in the pressed down state (ON) and the detection result of the fixation detection sensor 26b is in the pressed down state (ON) in step S19-1, the process proceeds to step S19-2.

In step S19-2, the controller 3 determines that this state is an attachment completed state.

In step S19-3, the controller 3 allows continuous use of the medical system 1A, and the process returns to step S19-1.

In a case other than the detection result of the attachment detection sensor 30a is in the pressed down state (ON) and the detection result of the fixation detection sensor 26b is in the pressed down state (ON) in step S19-1, the process proceeds to step S19-4.

In step S19-4, the controller 3 determines that this state is an attachment error state.

In step S19-5, the controller 3 makes an emergency stop of the medical system 1A.

In step S19-6, the controller 3 prohibits control input to the first to ninth driving sources (M11 to M33). At this time, for example, control input to components and units, which can be factors of failure, such as a motor (not shown) that moves the movable stage 2a, may also be prohibited.

In step S19-7, the controller 3 notifies that the medical system 1A is brought into an emergency stop due to an attachment error state with, for example, the monitor 4, and ends the flowchart.

As described above, a further detailed state, specifically, an attachment completed state, a temporarily attached state, an unattached state, and an attachment error state, regarding attachment of the catheter unit 100 with the base unit 200 can be notified based on the detection result of the attachment detection sensor 30a and the detection result of the fixation detection sensor 26b.

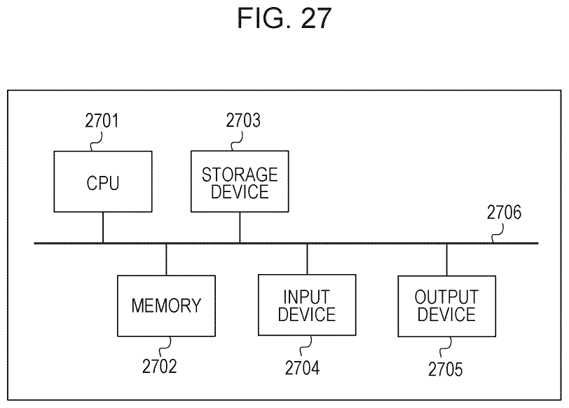

FIG. 27 is an example of the hardware configuration for implementing an information processing apparatus according to the present invention. The controller 3 includes a CPU 2701, a memory 2702, a storage device 2703, an input device 2704, and an output device 2705 each are connected to one another by a bus 2706.

The CPU 2701 runs a program stored in the storage device 2703. Thus, the process of the above-described flowchart is executed. In this way, the CPU 2701 runs a program to implement functions of acquisition means, notification means, and control prohibition means according to the present invention. The memory 2702 temporarily stores a program and/or data that the CPU 2701 reads out from the storage device 2703. The memory 2702 is also used as an area for the CPU 2701 to run various programs. The storage device 2703 stores an operating system (OS), various programs, and various pieces of data. The input device 2704 is a functional unit that receives input from a user and is, for example, a keyboard or a mouse. The output device 2705 outputs information input by the input device 2704 and an execution result of a program run by the CPU 2701.

The present invention has been described together with the embodiments; however, the above-described embodiments only illustrate specific examples in carrying out the present invention, and the technical scope of the present invention should not be interpreted restrictively. The present invention may be implemented in various forms without departing from the technical idea or main characteristics of the present invention. The present invention may be implemented in various forms without departing from the technical idea or main characteristics of the present invention.

### Other Embodiments

The present invention can be implemented by processing of supplying a program for implementing one or more functions of the above-described embodiments to a system or apparatus via a network or storage medium, and causing one or more processors in the computer of the system or apparatus to read out and execute the program. Alternatively, embodiments of the present invention may be implemented by a circuit (for example, ASIC) that implements one or more functions.

Embodiments of the present invention are not limited to the above-described embodiments. Various changes or modifications are applicable without departing from the spirit and scope of the present invention. Therefore, the following claims are attached to show the scope of the present invention.

According to the present invention, it is possible to assist in attaching/detaching work in a continuum robot configured such that a bendable unit is detachably attachable to a base unit.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A continuum robot comprising:
a base unit including a driving source and a coupling portion connected to the driving source;
a bendable unit detachably attached to the base unit, the bendable unit including a bend portion configured to bend and a held portion coupled to the coupling portion and configured to move so as to bend the bend portion, the bendable unit being detachably attachable to the base unit;
an operating portion movable between a fixed position where the coupling portion and the held portion are fixed to each other and a position where the coupling portion and the held portion are not fixed to each other, in a state where the bendable unit and the base unit are attached; and
fixation detection means configured to detect whether the operating portion is at the fixed position.

2. The continuum robot according to claim 1, further comprising attachment detection means configured to detect whether the bendable unit and the base unit are attached to each other.

3. The continuum robot according to claim 1, comprising:
a plurality of the driving sources;
a plurality of the coupling portions respectively coupled to the plurality of driving sources; and
a plurality of the held portions respectively coupled to the plurality of coupling portions, wherein
when the operating portion is moved to the fixed position, the plurality of coupling portions and the plurality of held portions are respectively fixed to each other.

4. An information processing apparatus comprising:
acquisition means configured to acquire a detection result of the fixation detection means from the continuum robot according to claim 1; and
notification means configured to notify that attachment of the bendable unit with the base unit is complete or attachment of the bendable unit with the base unit is not complete, in accordance with the detection result of the fixation detection means.

5. The information processing apparatus according to claim 4, wherein, in use of the continuum robot, the notification means is configured to, in a case where the fixation detection means detects that the operating portion is not at the fixed position, notify an attachment error.

6. The information processing apparatus according to claim 4, further comprising control prohibition means configured to prohibit control over the driving source.

7. An information processing apparatus comprising:
acquisition means configured to acquire a detection result of the attachment detection means and a detection result of the fixation detection means from the continuum robot according to claim 2; and
notification means configured to notify an attachment completed state, a temporarily attached state, an unattached state, or an attachment error state regarding attachment of the bendable unit with the base unit based on the detection result of the attachment detection means and the detection result of the fixation detection means.

8. An information processing apparatus comprising:
acquisition means configured to acquire a detection result of the attachment detection means and a detection result of the fixation detection means from the continuum robot according to claim 2; and
notification means configured to, in a case where the attachment detection means detects that the bendable unit and the base unit are attached to each other and the fixation detection means detects that the operating portion is at the fixed position, notify that the bendable unit and the base unit are in an attachment completed state.

9. The information processing apparatus according to claim 8, wherein the notification means is configured to, in a case where the attachment detection means detects that the bendable unit and the base unit are attached to each other and the fixation detection means detects that the operating portion is not at the fixed position, notify that the bendable unit and the base unit are in a temporarily attached state.

10. The information processing apparatus according to claim 8, wherein the notification means is configured to, in a case where the attachment detection means detects that the bendable unit and the base unit are not attached to each other and the fixation detection means detects that the operating portion is not at the fixed position, notify that the bendable unit and the base unit are in an unattached state.

11. The information processing apparatus according to claim 8, wherein the notification means is configured to, in a case where the attachment detection means detects that the bendable unit and the base unit are not attached to each other and the fixation detection means detects that the operating portion is at the fixed position, notify an attachment error state.

12. The information processing apparatus according to claim 7, further comprising control prohibition means configured to prohibit control over the driving source.

13. An information processing method comprising:
acquiring a detection result of the fixation detection means from the continuum robot according to claim 1; and
notifying that attachment of the bendable unit with the base unit is complete or attachment of the bendable unit with the base unit is not complete, in accordance with the detection result of the fixation detection means.

14. An information processing method comprising:
acquiring a detection result of the attachment detection means and a detection result of the fixation detection means from the continuum robot according to claim 2; and
notifying an attachment completed state, a temporarily attached state, an unattached state, or an attachment error state regarding attachment of the bendable unit to the base unit based on the detection result of the attachment detection means and the detection result of the fixation detection means.

15. An information processing method comprising:
acquiring a detection result of the attachment detection means and a detection result of the fixation detection means from the continuum robot according to claim 2; and
in a case where the attachment detection means detects that the bendable unit and the base unit are attached to each other and the fixation detection means detects that the operating portion is at the fixed position, notifying that the bendable unit and the base unit are in an attachment completed state.

16. A non-transitory storage medium recording a program for causing a computer to function as the means of the information processing apparatus according to claim 4.
